(19) United States
(12) Patent Application Publication
(10) Pub. No.: US 2014/0328765 A1
(43) Pub. Date: Nov. 6, 2014

(54) T1-T2 DUAL MODAL MRI CONTRAST AGENTS

(71) Applicant: INDUSTRY-ACADEMIC COOPERATION FOUNDATION, YONSEI UNIVERSITY, Seoul (KR)

(72) Inventors: Jin Woo CHEON, Seoul (KR); Jin-Sil CHOI, Seoul (KR)

(21) Appl. No.: 14/336,827

(22) Filed: Jul. 21, 2014

Related U.S. Application Data
(63) Continuation-in-part of application No. 13/059,295, filed on May 5, 2011, now abandoned.

(30) Foreign Application Priority Data
Aug. 21, 2008 (KR) 10-2008-0081844

(51) Int. Cl. A61K 49/18

(52) U.S. Cl. CPC A61K 49/1824
USPC 424/9.32; 424/9.3

(57) ABSTRACT

The present invention relates a T1-T2 dual-modal MRI (magnetic resonance imaging) contrast agent, comprising (a) a first layer consisting of T1 contrast material; (b) a second layer consisting of T2 contrast material; and (c) a separating layer which is present in a space between the first layer and the second layer, and inhibits a reciprocal interference between T1 contrast material and T2 contrast material, and a heat-generating composition and a drug delivery composition having the same. The T1-T2 dual-modal contrast agent of the present invention may generate both T1 and T2 signal and thus observe the signal complementarily, resulting in accurate diagnosis through reduction of misdiagnosis. Further, T1 and T2 MR imaging may be simultaneously obtained by simple operation within the same MR imaging device, enabling to remarkably reduce a diagnosis time and diagnosis cost. In addition, the particle constituting the T1-T2 dual-modal contrast agent of the present invention may be applied to hyperthermia and drug delivery systems.

### CROSS-REFERENCE TO RELATED APPLICATIONS

This application is a continuation-in-part of U.S. Ser. No. 13/059,295, filed May 5, 2011, which is a U.S. national stage filing under 35 U.S.C. 371 of PCT/KR2009004683, filed Aug. 21, 2009, which claims priority from KR 10-2008-0081844, filed Aug. 21, 2008. Each of the prior applications is incorporated herein by reference.

### BACKGROUND OF THE INVENTION

1. Field of the Invention

The present invention relates to a T1-T2 dual modal MRI contrast agent, a heat-generating composition, and a drug-delivery composition.

2. Description of the Related Art

Nanomaterial exhibits new physiochemical characteristics different from bulk material when its size is reduced to a nano-scale particle. The intensive researches for the nanomaterials permit nanomaterials to be precisely controlled in their composition and shape as well as the size, realizing the physiochemical properties in a nano-region. Current nanotechnologies have been rapidly developed in a variety of applications and widely classified into three fields: i) a technology for synthesizing a novel micro-sized substance and material by a nanomaterial, ii) a technology for preparing a device for certain performance by combining or fabricating nano-sized materials in a nano-device; and iii) a technology in which nanotechnology, called a nano-bio, is grafted into biotechnology.

Among various nanoparticles, magnetic nanoparticles can be extensively used in the nano-bio technology including biomolecule isolation, a magnetic resonance imaging (MRI) diagnosis, a magnetism-bio sensor (e.g, a giant magnetoresistance sensor), a microfluid system, drug/gene delivery system and a magnetic hyperthermia.

In particular, the magnetic nanoparticle can serve as diagnostic agents for magnetic resonance imaging.

MRI measures a nuclear spin relaxation of hydrogen atoms of water molecules, providing T1 and T2 images. MRI contrast agents are classified into a T1 contrast agent and a T2 contrast agent, allowing for the amplification of T1 or T2 signals. Following the nuclear spin is excited, T1 and T2 refer to a spin-lattice relaxation time and a spin-spin relaxation time in MRI, respectively, and contribute to different imaging effects from each other. T1 contrast agents are composed of paramagnetic materials generating spin-lattice relaxation. Generally, a bright or positive contrast effect is obtained in the presence of T1 contrast agents as compared to water. Gd-chelate compounds may be mainly used as T1 contrast agents. A commercially available Magnevist (Schenng, Germany) used for MR imaging contains Gd-DTPA (Gd-diethylene triamine pentaacetic acid). In addition, it has been reported that several materials such as Gd₂O₃ (C. Riviere et al. J. Am. Chem. Soc. 2007, 129, 5076) and MnO (T. Hyeon et al. Angew. Chem. Int. Ed. 2007, 46, 5397) are used as T1 contrast agents.

In contrast, superparamagnetic nanoparticles such as iron oxide nanoparticles have been used as prevailing T2 contrast agents. Under external magnetic field, such magnetic nanoparticles are magnetized and induced an additional magnetic field. As a result, a spin-spin relaxation process of the nuclear spins of hydrogen atoms of nearby water molecules is influenced to amplify MRI signals, thereby showing a dark or negative contrast effect compared to water. T2 contrast agents predominantly used in the art include Feridex, Resovist and Combidex that contain iron oxide components. Recently, MEIO (magnetism engineered iron oxide) in which a portion of iron oxide components is substituted to greatly enhance contrast effects has been developed as a promising T2 contrast agent (J. Cheon et al. Nature Medicine 2007, 13, 95).

In MRI, the T1 image mode has excellent resolution between tissues due to its high signal intensity (bright signal), resulting in discriminating an anatomical structure in detail. It is also advantageous that T1 imaging is useful for determining the presence or absence of bleeding in lesion because high signal intensity is characteristically shown in subacute bleeding (4-14 days after bleeding). On the contrary, tissue resolution of T2 imaging is lower than that of T1 imaging, but it has an advantage that the lesion is detected easier in the T2 imaging than in the T1 imaging because most lesion tissues exhibited higher signal intensity in the T2 imaging than in the normal imaging (D. W. McRobbie et al. MRI from Picture to Proton Cambridge, 2003).

The magnetic resonance imaging amplified by contrast agent may be effective for a disease diagnosis or imaging a living phenomenon in molecular or cellular level. Using a dual modal contrast agent representing beneficial contrast effects in both T1 imaging with high tissue resolution and T2 imaging with high feasibility on diagnosing a lesion, it is expected that a pathological diagnosis is accurately performed. However, T1-T2 dual modal MRI contrast agent has not been developed yet up to date because T2 contrast material interferes with a magnetic property of T1 contrast material, quenching T1 signal. In general, the magnetic material (e.g., ferromagnetic, ferrimagnetic or supermagnetic material) having T2 contrast effect has its original magnetism or easily generates the induced magnetic field by external magnetic field. Where the paramagnetic materials having T1 contrast effect are closely located so as to be influenced by the magnetic field, the changes of their spin order and spin relaxation is generated (for example, a T1 paramagnetic material exhibits antiparallel spin ordering toward an opposite direction with spin of a ferromagnetic, ferrimagnetic or supermagnetic material (Y. Oda et al, Journal of Physical Society of Japan, 2008, 77, 073704-1; J. P. Liu et al. Journal of Applied Physics 2003, 94, 6673)). Therefore, in the material linked by contacting the T1 and T2 MRI contrast agent, the magnetic field produced by T2 contrast material affects the spin relaxation of T1 contrast material, and thus the spin-lattice relaxation of water via T1 contrast material is reduced, resulting in reduction of T1 contrast effect (T1 signal quenching).

Likewise, signal quenching according to a distance is occurred in fluorescence. FRET (fluorescence resonance energy transfer) used for a biological detection frequently is a method which detects a DNA, a peptide or a protein by observing fluorescence changes according to a distance. FRET is generated when a fluorescence-acceptor material is adjacent to a fluorescence-donor material having emission energy in a similar region with absorption energy of the fluorescence-acceptor material. The electron released from the fluorescence-donor material is absorbed into fluorescence-acceptor material, and then the fluorescence of fluorescence-donor material is reduced (quenching). The quenching of the fluorescence is varied depending on the distance between the fluorescence-donor material and the fluorescence-acceptor material (H. Mattoussi et al. Nature Mater. 2006, 5, 581).

On the other hand, the intensive studies to integrate both MRI contrast agents have been progressed, but no researches successfully established the dual modal MRI contrast agent because the signal quenching of T1 contrast material by T2 contrast material is not effectively controlled. Few researches on the dual modal MRI contrast agent are as follows.

WO 00/09170A1 discloses a simultaneous use of T1 and T2 contrast agent for imaging a blood vessel. However, the technique disclosed in the specification focused on not a complementary effect between both contrast agents, but an improvement of a diagnostic efficiency by comparing T1 imaging with T2 imaging of MRI after injection into animals using simple mixture of T1 and T2 contrast agent. It is difficult to obtain the corresponding information simultaneously using such diagnostic method because both contrast agents are different in dynamic action in the living body and the present time in the same region.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY OF THE INVENTION

The present inventors have made intensive researches to develop a T1-T2 dual modal MRI contrast agent in which T1 and T2 signals could be generated in a single particle. As results, we have discovered that a single particle prepared to contain a separating layer introduced into a space between a T1 contrast material and a T2 contrast material permits to control a signal quenching caused from a magnetic interference effect on T1 contrast materials by T2 contrast materials, and is able to exhibit T1 and T2 contrast effects in a clinically practical fashion and to obtain T1 and T2 MR images simultaneously in conventional MRI devices.

Accordingly, it is an object of this invention to provide an effective T1-T2 dual modal MRI contrast agent by preparing a single nanoparticle in which T1 and T2 contrast effect, and a relative contrast effect thereof are artificially controlled.

It is another object of this invention to provide a method for providing T1 and T2 images of an internal region of a patient.

It is still another object of this invention to provide a heat-generating composition.

It is further still another object of this invention to provide a drug-delivery composition.

Other objects and advantages of the present invention will become apparent from the following detailed description together with the appended claims and drawings.

### BRIEF DESCRIPTION OF THE DRAWINGS

The file of this patent or application publication contains at least one drawing executed in color. Copies of this patent or application publication with color drawing(s) will be provided by the Office upon request and payment of the necessary fee.

FIG. 1 represents a structure of a preferable T1-T2 dual modal MRI contrast agent which includes all structures having a separating layer in a space between T1 contrast material and T2 contrast material.

FIGS. 2a-2d represent a core-separating layer-shell structure as a preferable example having the T1-T2 dual modal MRI contrast agent structure of the present invention. The separating layer may consist of a hard shell of a solid shape (FIG. 2a), a linkage by Van der Waals force (FIG. 2b), a layer-by-layer (LBL) by an electrostatic attraction (FIG. 2c), or a porous structure (FIG. 2d).

FIG. 3 is transmission electron microscope (TEM) images of a nanoparticle useful in the T1-T2 dual modal MRI contrast agent of the present invention. The TEM image of nanoparticles is as follows: (a), Fe₃O₄ nanoparticles; (b), MnFe₂O₄ nanoparticles; (c), CoFe₂O₄ nanoparticles; (d), NiFe₂O₄ nanoparticles; (e-h), ZnₓMn₁₋ₓFe₂O₄ (x=0.2, 0.3, 0.4, 0.8) nanoparticles; (i-l), ZnₓFe₁₋ₓFe₂O₄ (x=0.2, 0.3, 0.4, 0.8) nanoparticles; (m), FePt nanoparticles; (n), Gd₂O₃ nanoparticles; (o), Dy₂O₃ nanoparticles; (p), Ho₂O₃ nanoparticles. (q), a schematic diagram representing sheet-typed nanoparticles observed at various angles. The sheet-typed nanoparticles were observed as a sphere shape in a horizontal face and a bar shape in a longitudinal face.

FIG. 4 represents the TEM images of magnetic nanoparticle, MnFe₂O₄@SiO₂, coated with SiO₂ separating layer at various thickness (4, 12, 16, 20 nm). The thickness of SiO₂ used in the separating layer may be varied depending on the amount of a reactant used.

FIG. 5 represent the TEM images of MnFe₂O₄@SiO₂@Gd₂O(CO₃)₂.H₂O having the core-separating layer-shell structure prepared according to the present method. Each 15 nm of MnFe₂O₄ nanoparticle and about 1.5 nm of Gd₂O(CO₃)₂.H₂O nanoparticle is used as the core and the shell. SiO₂ thickness may be variously controlled in a range of from 4 nm to 20 nm (4, 8, 12, 16, 20 nm).

FIG. 6 is a XRD (X-ray diffraction) graph from the shell of the core-shell structure prepared according to the present method, representing a crystal structure of the shell is consistent with that of Gd₂O(CO₃)₂.H₂O (JCPDS #: 43-0604).

FIG. 7 represents results of MnFe₂O₄@SiO₂@Gd₂O(CO₃)₂.H₂O nanoparticle having SiO₂ separating layer at various thickness (4, 8, 12, 16, 20 nm): (a) T1 image; (b) a comparative graph of T1 relaxivity coefficient (r1); and (c) a graph of a relative T1 signal quenching effect compared with H₂O (standard material). In T1 image of thin SiO₂ separating layer, low T1 effect is observed as compared with H₂O (standard material) due to T1 signal quenching caused from the magnetic interference effect with T1 contrast material by T2 contrast material. Depending that the separating layer is thick, T1 signal quenching is reduced whereas T1 signal is increased. It could be appreciated that T1 contrast effect represented the most excellent effect in 16 nm of SiO₂ separating layer and also the reduced T1 signal quenching had the same tendency.

FIG. 8 represents results of MnFe₂O₄@SiO₂@Gd₂O(CO₃)₂.H₂O nanoparticle having the SiO₂ separating layer at various thickness (4, 8, 12, 16, 20 nm): (a) T2 MR image; (b) a comparative graph of T2 relaxivity coefficient (r2), and (c) a graph representing changes of r2/r1. All nanoparticles had the excellent T2 contrast effects as compared with H₂O, but exhibited some changes in comparison with a MnFe₂O₄ nanoparticle in which the separating layer or shell layer is not coated. It could be appreciated that all nanoparticles have a relatively high T2 contrast effect.

FIG. 9 is T1 or T2 image of nanoparticle having the core-separating layer-shell structure prepared according to the present method. The size of nanoparticle used as the core is 15 nm except FePt (6 nm), and T1 contrast material consisting of the shell is prepared with a size of about 1.5 nm. The SiO₂ separating layer was coated with the thickness of 16 nm as shown in FIG. 6. (a) FePt@SiO₂@Gd₂O(CO₃)₂.H₂O, (b) Fe₃O₄@SiO₂@Gd₂O(CO₃)₂.H₂O, (c) CoFe₂O₄@SiO₂@Er₂O(CO₃)₂.H₂O, (d) CoFe₂O₄@SiO₂@Gd₂O(CO₃)₂.H₂O, (e) CoFe₂O₄@SiO₂@Dy₂O(CO₃)₂.H₂O, (f) MnFe₂O₄@SiO₂-DTPA-Gd, (g) MnFe₂O₄@PS-b-PMMA@Gd, (h) MnFe₂O₄@SA-DTPA-Gd and (i) H₂O. All illustrative nanoparticles having the core-separating layer-shell structure represented more light signals in T1 image than those in T1 image of water, and more dark signals in T2 image than those in T2 image of water. In other words, the core-separating layer-shell-type contrast agent of the present invention significantly represented the increased T1 and T2 signals without respect to its composition.

FIG. 10 is T1 and T2 image in liver tissue using MnFe₂O₄@SiO₂@Gd₂O(CO₃)₂.H₂O contrast agent having the core-separating layer-shell structure. Magnetic resonance imaging (MRI) was measured at 1 hr pre- and post-injection of nanoparticles, respectively. It was demonstrated that the signals (T1 and T2 signal) in liver at post-injection were increased in comparison with those in liver at pre-injection.

FIG. 11 represents T1 and T2 image in cancer tissue using MnFe₂O₄@SiO₂@Gd₂O(CO₃)₂.H₂O contrast agent having the core-separating layer-shell structure. It was demonstrated that the signals (T1 and T2 signal) in cancer tissue at post-injection were increased in comparison with those in cancer tissue at pre-injection.

### DETAILED DESCRIPTION OF THIS INVENTION

In one aspect of this invention, there is provided a T1-T2 dual-modal MRI (magnetic resonance imaging) contrast agent, comprising (a) a first layer containing a T1 contrast material; (b) a second layer containing a T2 contrast material; and (c) a separating layer, located between the first layer and the second layer, to prevent a reciprocal interference effect between the T1 contrast material and the T2 contrast material.

In another aspect of this invention, there is provided a method for providing T1 and T2 images of an internal region of a patient, which comprises the steps of: (a) administering to the patient a diagnostically effective amount of the T1-T2 dual-modal MRI contrast agent; and (b) scanning the patient using a magnetic resonance imaging to obtain visible T1 and T2 images of the region.

The present inventors have made intensive researches to develop a T1-T2 dual modal MRI contrast agent generating T1 and T2 signal in a single particle. For this purpose, a separating layer was introduced into a space between a T1 contrast material and a T2 contrast material so as to control a signal quenching caused from a magnetic interference effect with T1 contrast material by T2 contrast material in the senses that the quenching of a fluorescent signal is depending on the distance as described above in FRET. Using the separating layer, the magnetic interference effect with T1 contrast material by T2 contrast material may be effectively inhibited. Thus, the present inventors have demonstrated that the single particle may generate T1 and T2 signal available on a clinic and obtain T1 and T2 MR imaging simultaneously through conventional MRI.

To achieve the functionality of the above-described T1-T2 dual-modal MRI contrast agent, the present invention constructs the T1-T2 dual-modal MRI contrast agent consisting of at least three parts: (a) the first layer containing a T1 contrast material; (b) the second layer containing a T2 contrast material; and (c) a separating layer located between the first layer and the second layer. The separating layer with an appropriate rigidity stably maintains the distance between the first layer and the second layer, inhibiting the magnetic interference effect between T1 contrast material and T2 contrast material efficiently. The T1-T2 dual-modal MRI contrast agent of the present invention may be variously fabricated depending on the number of the first layer, the second layer and the separating layer.

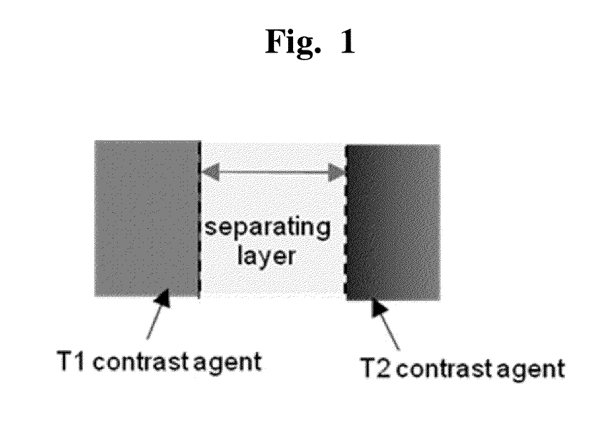

The T1-T2 dual-modal MRI contrast agent of the present invention includes all materials having the structure in which the separating layer is located in a space between T1 contrast material and T2 contrast material. FIG. 1 is a schematic diagram about a basic structure of the contrast agent of this invention. Preferably, the structure of the present T1-T2 dual-modal contrast agent includes the separating layer between T1 contrast material and T2 contrast material as shown in FIG. 1 and thus the T1-T2 dual-modal MRI contrast agent of the present invention includes all materials having the structure in which the separating layer is located between T1 contrast material and T2 contrast material. Preferably, the T1-T2 dual-modal MRI contrast agent of this invention may utilize various materials including, but not limited to, a global shape, a bar shape, a column shape, a sheet shape, a layered structure, a dumbbell shape, a core-satellite structure, a porous structure, a host-guest structure and a modified structure thereof. More preferably, the T1-T2 dual modal MRI contrast agent of this invention is the core-separating layer-shell structure or the modified structure thereof, in which each T1 or T2 contrast agent is in the core or shell, and the separating layer is in the space between T1 contrast agent and T2 contrast agent. The core-separating layer-shell structure may be constructed into various shapes or structures as described above.

In the present contrast agent having the core-shell structure, the separating layer is introduced into the space between the core and the shell. Preferably, the T1-T2 dual-modal MRI contrast agent of this invention has a core-shell structure including (a) a core containing a T1 contrast material or a T2 contrast material; (b) a shell containing a T2 contrast material or a T1 contrast material; and (c) a separating layer, located between the core and the shell, to prevent a reciprocal interference effect between the T1 contrast material and the T2 contrast material, and wherein the core and shell include a different type of the contrast materials from each other.

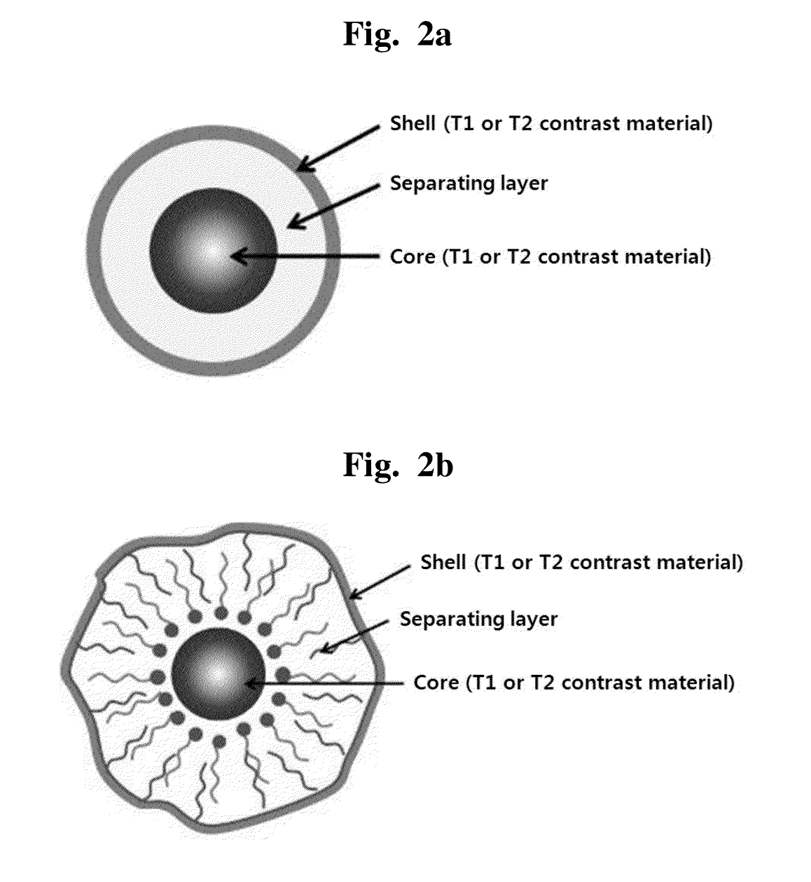

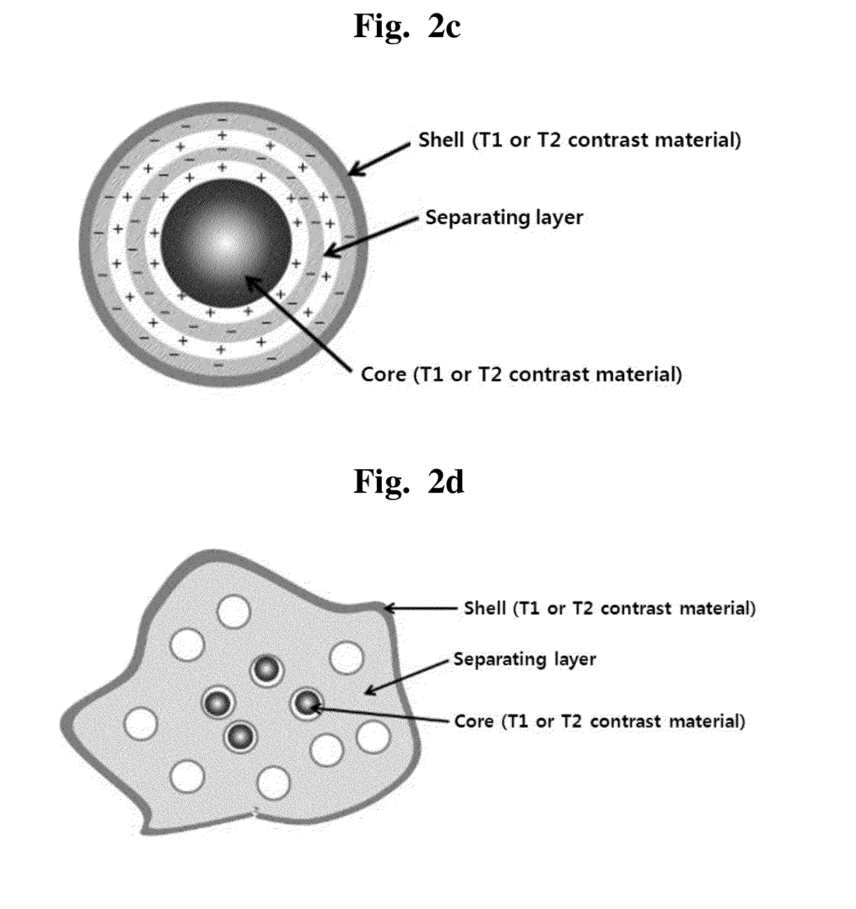

The separating layer is attached with or is bound to the T1 or T2 contras material by an ionic bond, an electrostatic interaction, a coordination bond, a hydrophobic interaction, a hydrogen bond, a covalent bond, a hydrophilic interaction or a Van der Waals force. Preferably, the separating layer grows on the surface of the first layer or the second layer attached, or forms the layer-by-layer (LBL) structure by an electrostatic attraction around the core material or builds up the porous structure. In FIG. 2, various types of the core-separating layer-shell structure are shown as an example of the T1-T2 dual-modal contrast agent. FIG. 2a represents that the separating layer having the core-separating layer-shell structure grows on the core material or is attached to the core material and thus is located between the T1 contrast material and the T2 contrast material, maintaining the distance between both materials effectively. FIG. 2b exhibits a modified example of the conventional core-separating layer-shell structure. For example, the material forming the micelle structure such as surfactant in the adjacent core forms the separating layer by a hydrogen bond, a hydrophilic interaction, a hydrophobic interaction or a Van der Waals force. In addition, the present contrast agent, as shown in FIG. 2c, has the core-separating layer-shell structure formed by a layer-by-layer (LBL) fabrication in which the separating layer (preferably containing organic materials) forms the layer-by-layer (LBL) structure by an electrostatic attraction around the core material, or as shown in FIG. 2d, has a structure in which T1 contrast materials or T2 contrast materials are contained in the center of the separating layer having a porous structure, and the other type of contrast materials is included in the outer part or coated on the surface of the separating layer.

To generate T1 and T2 effect simultaneously, the considerable point is a thickness of the separating layer. In the magnetic nanoparticle used as a T2 contrast material, the strength of the magnetic field is reduced depending on inverse proportion to about a cube of the distance from the nanoparticle (M. M. Miller et al. Journal of Magnetism and Magnetic Materials 2001, 225, 138). Therefore, the separating layer has to simultaneously (a) minimize the quenching of a T1 contrast signal by ensuring a sufficient distance so that the T1 contrast material is a little affected by the magnetic field of the T2 contrast material and (b) possess a thickness to maintain an effective T2 contrast effect by not being long distance between T2 contrast material and the substance where the separating layer has the structure enclosing the T2 contrast material. According to the preferable embodiment, the separating layer has the thickness to prevent the magnetic interference effect with T1 contrast material by T2 contrast material, and more preferably, the separating layer is thicker than 1/1,000 of a radius of T2 contrast material, and most preferably the separating layer is thicker than 1/100 of a radius of T2 contrast material. The thickness of the separating layer is not limited, but has preferably not more than 1,000 times of a radius of the core contrast material, and more preferably not more than 100 times of a radius of the core contrast material.

According to the preferable embodiment, the separating layer has the thickness in the range of 8-20 nm, more preferably 12-20 nm, and most preferably 15-18 nm.

According to the preferable embodiment, the ratio of an average thickness of the separating layer and an average size of the shell ranges from 8:15 to 4:3, more preferably 4:5 to 4:3, and most preferably 11 to 6:5.

The separating layer of this invention may realize the dual modal MRI contrast agent only in the presence of the separating layer having the thickness enough to partially prevent the T1 signal quenching between the T2 contrast material and the T1 contrast material although the distance of all directions between the T2 contrast material and the T1 contrast material is unequal.

In this case, it is preferable that the separating layer consists of a material with some rigidity. The rigid separating layer functions to divide the T1 contrast material with the T2 contrast material in a stable manner. For example, the T1 contrast material and T2 contrast material was not effectively separated because the distance between two materials is diminished by folding or curving of a chemical molecule in separation of the T1 contrast material and the T2 contrast material using a simple chemical molecule. Thus, the distance between the T1 contrast material and the T2 contrast material has to be efficiently maintained by using the materials with more rigidity.

As an illustrative example of the T1-T2 dual modal contrast agent, the T1 contrast material and the T2 contrast material is located in the core or shell of the core-separating layer-shell structure, in which different types of contrast material are contained. For instance, when the core contains T2 contrast materials, T1 contrast materials may be involved in the shell. On the contrary, T2 contrast materials may be involved in the shell when the core contains T1 contrast materials. For effective operation of the T1-T2 dual modal contrast agent, the conditions are as follows: a) each magnetic nanoparticles used in T2 or T1 contrast agent have to exhibit an excellent magnetic property; and b) the surface area of materials used as T1 contrast agents is so broad to have much more opportunities for direct contact with water molecules. Therefore, it is preferable that each core and shell is composed of T2 material and T1 material.

Although the contrast materials constituting the shell would not homogenously coat the surface of all separating layers and include a carrier structure (example: a layered structure, a porous structure) having the space capable of attaching the separating layer or accommodating the contrast material, it may be used in the dual modal MRI contrast agent. The above-mentioned core may be composed of one or more contrast materials.

Preferably, the separating layer of this invention consists of a material without magnetic property or with weak magnetic property not to affect T1 or T2 contrast effect.

The material used in the separating layer has a rigid structure and thus includes an inorganic material, an organic material or a multi-component hybrid structure thereof to firmly separate T1 contrast materials from T2 contrast materials.

Preferably, the inorganic material capable of being used in the separating layer includes several inorganic elements (M), an inorganic chalcogen compound, an inorganic pnicogen compound, an inorganic carbon compound, an inorganic boron compound, a ceramic material, a metal complex compound or a multi-component hybrid structure thereof.

The inorganic element (M) is one or more elements selected from the group consisting of Group 1 metal elements, Group 2 metal elements, transition metal elements, Group 13-17 elements, Lanthanide metal elements and Actinide metal elements, and most preferably, Group 1 metal elements (Li, Na, K, Rb), Group 2 metal elements (Be, Mg, Ca, Sr, Ba), Group 13 elements (B, Al, In, Tl), Group 14 elements (C, Si, Ge, Sn, Pb), Group 15 elements (P, As, Sb, Bi), Group 16 elements (S, Se, Te, Po), Group 17 elements (I), transition metal elements (Sc, Ti, V, Zn, Y, Zr, Nb, Mo, Pd, Ag, Cd, W, Re), Lanthanide metal elements (Ce, Pr, Nd, Pm, Sm, Eu, Lu) and Actinide metal elements, or a multi-component hybrid structure thereof.

According to a preferable embodiment, the inorganic chalcogen compound includes MₓA_{y} (M=one or more elements selected from the group consisting of Group 1 metal elements, Group 2 metal elements, Group 13-15 elements, Group 17 elements, transition metal elements, Lanthanide metal elements and Actinide metal elements; A is one or more elements selected from the group consisting of O, S, Se, Te and Po; 0<x≦16, 0<y≦8), or a multi-component hybrid structure thereof. More preferably, the inorganic chalcogen compound includes MₓA_{y} (M=one or more elements selected from the group consisting of Group 1 metal elements (Li, Na, K, Rb), Group 2 metal elements (Be, Mg, Ca, Sr, B), Group 13 elements (B, Al, In, Tl), Group 14 elements (C, Si, Ge, Sn, Pb), Group 15 elements (P, As, Sb, Bi), Group 17 elements (F, Cl, Br, I), transition metal elements (Sc, Ti, V, Zn, Y, Zr, Nb, Mo, Pd, Ag, Cd, W, Re), Lanthanide metal elements (Ce, Pr, Nd, Pm, Sm, Eu, Lu) and Actinide metal elements; A is one or more elements selected from the group consisting of O, S, Se, Te and Po; 0<x≦16, 0<y≦8), or a multi-component hybrid structure thereof, and much more preferably MₓA_{y} (M=one or more elements selected from the group consisting of Group 1 metal elements (Li, Na, K, Rb), Group 2 metal elements (Be, Mg, Ca, Sr, Ba), Group 13 elements (B, Al, In, Tl), Group 14 elements (C, Si, Ge, Sn, Pb), Group 15 elements (P, As, Sb, Bi), Group 17 elements (F, Cl, Br, I), transition metal elements (Sc, Ti, V, Zn, Y, Zr, Nb, Mo, Pd, Ag, Cd, W, Re), Lanthanide metal elements (Ce, Pr, Nd, Pm, Sm, Eu, Lu) and Actinide metal elements; 0<x≦16, 0<y≦8), or a multi-component hybrid structure thereof.

Preferably, the inorganic pnicogen compound includes MₓA_{z} (M=one or more elements selected from the group consisting of Group 1 metal elements, Group 2 metal elements, Group 13 elements, Group 14 elements, Group 16 elements, Group 17 elements, transition metal elements, Lanthanide metal elements and Actinide metal elements; A is one or more elements selected from the group consisting of N, P, As, Sb and Bi; 0<x≦24, 0<z≦8), or a multi-component hybrid structure thereof, and more preferably MₓA_{z} (M=one or more elements selected from the group consisting of Group 1 metal elements (Li, Na, K, Rb), Group 2 metal elements (Be, Mg, Ca, Sr, Ba), Group 13 elements (B, Al, In, Tl), Group 14 elements (C, Si, Ge, Sn, Pb) Group 16 elements (S, Se, Te, Po), Group 17 elements (F, Cl, Br, I), transition metal elements (Sc, Ti, V, Zn, Y, Zr, Nb, Mo, Pd, Ag, Cd, W, Re), Lanthanide metal elements (Ce, Pr, Nd, Pm, Sm, Eu, Lu) and Actinide metal elements; A is one or more elements selected from the group consisting of N, P, As, Sb and Bi; 0<x≦24, 0<z≦8), or a multi-component hybrid structure thereof.

The inorganic carbon compound capable of being used in the separating layer includes MₓA_{z} (M=one or more elements selected from the group consisting of Group 1 metal elements, Group 2 metal elements, Group 13 elements, Group 15-17 elements, transition metal elements, Lanthanide metal elements and Actinide metal elements; A is one or more elements selected from the group consisting of C, Si, Ge, Sn and Pb; 0<x≦32, 0<z≦8), or a multi-component hybrid structure thereof.

The inorganic boron compound capable of being used in the separating layer includes MₓA_{z} (M=one or more elements selected from the group consisting of Group 1 metal elements, Group 2 metal elements, Group 14-17 elements, transition metal elements, Lanthanide metal elements and Actinide metal elements; A is one or more elements selected from the group consisting of B, Al, Ga, In and Tl; 0<x≦40, 0<z≦8), or a multi-component hybrid structure thereof.

Preferably, the ceramic material capable of being used in the separating layer includes the inorganic chalcogen material such as an inorganic oxide, and for example, titania, zirconia, silica, alumina, aluminate-containing inorganic compound, silicate-containing inorganic compound, zeolite, titanate-containing inorganic compound, ZnO, belemnite-containing inorganic compound, potassium phosphate-containing inorganic compound, calcite, apetite-containing inorganic compound, Sialon (silicon aluminium oxynitride), vanadate-containing inorganic compound, KTP (potassium titanyl phosphate)-containing inorganic compound, KTA (potassium titanyl Arsenate)-containing inorganic compound, borate-containing inorganic compound, fluoride-containing inorganic compound, fluorophosphate-containing inorganic compound, tungstate-containing inorganic compound, molybdate-containing inorganic compound, gallate-containing inorganic compound, selenide-containing inorganic compound, telluride-containing inorganic compound, niobate-containing inorganic compound, tantalate-containing inorganic compound, cuprite (Cu₂O), Ceria, bromelite (BeO), a porous material (example: MCM (mesoporous crystalline material)-41, MCM-48, SBA-15, SBA-16, a mesoporous or microporous material), or a multi-component hybrid structure thereof, but not limited to.

Preferably, the separating layer includes a metal complex compound. The metal complex compound refers all materials consisting of a center metal and a ligand bound to the metal coordinately, and particularly compound used in the separating layer is a complex compound composed of a center metal without the magnetic property and a coordination ligand. Preferably, the metal complex compound includes, but not limited to, MₓL_{y} (M=one or more elements selected from the group consisting of Group 1 metal elements, Group 2 metal elements, Group 13-17 elements, transition metal elements, Lanthanide metal elements and Actinide metal elements; L is one or more ligands capable of binding with a metal through a coordination bond; 0<x≦10, 0<y≦120), or a multi-component hybrid structure thereof.

More preferably, the metal complex compound capable of being used in the separating layer includes an organometallic compound, a metal organic framework (MOF) or a coordination polymer.

The organometallic compound is the metal complex compound in which a center metal is bound to a carbon of a coordination ligand.

The MOF is a multi-dimensional crystalline compound in which a rigid ligand is structurally bound to a center metal through a coordination bond. It is preferable to use the rigid structure as a separating layer due to its function to maintain the distance between a T1 contrast material and a T2 contrast material.

The coordination ligand is a metal complex compound with a multi-dimensional structure formed by a repetitive linkage between a metal and a ligand. The separating layer of the multi-dimensional structure may effectively separate the T1 contrast material from the T2 contrast material.

In addition, the organic material capable of being used in the separating layer is not particularly limited where the organic material with some rigidity functions to successively divide the T1 contrast material with the T2 contrast material in a stable manner.

The preferable organic material of the present invention includes a polymer, a polypeptide, a protein, a lipid, a nucleic acid or a chemical molecule.

The polymer capable of being used in the separating layer includes a synthetic polymer or a natural polymer.

The synthetic polymer includes any polymer which contains the functional group with rigidity, and preferably polyester, polyhydroxyalkanoate (PHAs), poly(α-hydroxy acid), poly(β-hydroxy acid), poly(3-hydroxybutyrate-co-valerate; PHBV), poly(3-hydroxproprionate; PHP), poly(3-hydroxyhexanoate; PHH), poly(4-hydroxy acid), poly(4-hydroxybutyrate), poly(4-hydroxyvalerate), poly(4-hydroxyhexanoate), poly(esteramide), polycaprolactone, polylactide, polyglycolide, poly(lactide-co-glycolide; PLGA), polydioxanone, polyorthoester, polyunhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(acrylate-co-styrene), pluronic copolymer, polyacrylamide, polyethylene glycol, poly(tyrosine carbonate), polycarbonate, poly(tyrosine arylate), polyalkylene oxalate, polyphosphagens, PHA-PEG (polyhydroxyalkanoate-polyethylene glycol), ethylene vinyl alcohol copolymer (EVOH), polyurethane, polystyrene, polyester, polyolefin, copolymer of polyisobutylene and ethylene-α-olefin, styrene-isobutylene-styrene triblock copolymer, acryl polymer and copolymer, vinyl halide polymer and copolymer, polyvinyl chloride, polyvinyl ether, polyvinyl methyl ether, polyvinylidene halide, polinylidene fluoride, polyvinylidene chloride, polyfluoroalkene, polyperfluoroalkene, polyacrylonitrile, polyvinyl ketone, polyvinyl aromatics, polystylene, polyvinyl ester, polyvinyl acetate, ethylene-methyl metacrylate copolymer, acrylonitrile-stylene copolymer, ABS [poly(acrylonitrile, butadiene, styrene)] resin, ethylene-vinyl acetate copolymer, polyamide, alkid resin, polyoxymethylene, polyimide, polyether, polyacrylate, polymethacrylate, polyacrylic acid-co-maleic acid, poly-L-lysine, polystyrene-polymethylmethylacrylate copolymer, poly-p-phenylene vinylene (PPV), polyalyl aminesulfonated polystylene, polyvinyl sulfate-polyvinyl amine copolymer, poly dialylmethylammoniurn chloride, poly-3,4-ethylenedioxythiophene (PEDOT), polyacrylamidosulfonic acid (PAMPS), or a derivative or polymer thereof, but not limited to.

The natural polymer capable of being used in the separating layer includes a carbohydrate, and preferably a polysaccharide. The most preferable example of the carbohydrate capable of being used in the separating layer includes, but not limited to, cellulose, starch, glycogen, chitosan, dextran, stachyose, schrodose, xylan, araban, hexosan, fructan, galactan, mannan, agaropectin, alginic acid, carrageenan, hemicelluloses, hypromellose, chitin, agarose, dextrin, carboxy methylcellulose, glycogen dextran, carbodextran, polysaccharide, cyclodextran, pullulan or a derivative thereof.

Another example of the preferable organic separating layer of the present invention is a peptide. The peptide may be effectively used as the separating layer due to a polymer structure consisting of several amino acids.

Still another example of the preferable organic separating layer of the present invention is a protein. Protein may be effectively used as the separating layer due to a polymer structure composed of more amino acids than peptides. The preferable example of protein includes a simple protein, a conjugated protein, a derived protein or an analog thereof. Much more preferable example of protein includes, but not limited to, a hormone, a hormone analog, an enzyme, an enzyme inhibitor, a signal-transducing protein or its part, an antibody or its part, a single chain antibody, a binding protein or its binding domain, an antigen, an attachment protein, a structural protein, a regulatory protein, a toxic protein, a cytokine, a transcription factor, a blood coagulation factor and a plant defense-inducible protein. Most preferably, protein capable of being used as the separating layer in the present invention includes, but not limited to, albumin, prolamine, glutenin, heparin, antibody (immunoglobulin), avidin, cytochrome, casein, myosin, glycinin, carotene, hemoglobin, myoglobin, flavin, collagen, streptavidin, protein A, protein G, protein S, lectin, selectin or angioprotein.

The chemical molecule capable of being used in the separating layer includes a material having a hydrophobic or a hydrophilic functional group. The chemical molecule forms a separating layer through binding to a core material via an electrostatic attraction, a hydrophobic interaction, an ionic bond, a hydrogen bond or a Van der Waals force. As a preferable example of the chemical molecule in the present invention, the hydrophobic functional group can be a linear or branched structure composed of chains containing 2 or more carbon atoms, and preferably an alkyl functional group (CₙHₘ; 0≦n≦20, 0≦m≦42), an alkene functional group (CₙHₘ; 0≦n≦30, 0≦m≦40) or an alkyn functional group (CₙHₘ; 0≦n≦20, 0≦m≦38), but not limited to. In addition, examples of the hydrophilic functional group include, but not limited to, the functional group being neutral at a specific pH, but being positively or negatively charged at a higher or lower pH such as —SH, —COOH, —NH₂, —OH, —PO₃H, —PO₄H₂, —SO₃H, —SO₄H, —NR₃⁺X⁻ (R═CₙHₘ, 0≦n≦16, 0≦m≦34, X═OH, Cl, Br), —CONH₂, —OPO₄H₂, —COSH, -hydrazone or the derivative thereof. Furthermore, preferable examples thereof include a polymer and a block copolymer, wherein monomers used include ethyleneglycol, acrylic acid, alkylacrylic acid, ataconic acid, maleic acid, fumaric acid, acrylamidomethylpropane sulfonic acid, vinylsulfonic acid, vinylphosphoric acid, vinyl lactic acid, styrenesulfonic acid, allylammonium, acrylonitrile, N-vinylpyrrolidone, N-vinylformamide, or the derivative or polymer thereof, but not limited to.

The preferable example of the above-described chemical molecule includes an amphiphilic surfactant containing both a hydrophobic and a hydrophilic functional group. Hydrophobic regions of ligands consisting of long carbon chains coat the surface of nanoparticles (the core in the present invention) synthesized in organic solvent. When amphiphilic ligands are added to the nanoparticle solution, the hydrophobic region of the amphiphilic material and the hydrophobic ligand on the nanoparticles are bound to each other through intermolecular interaction to form a separating layer. Further, the outermost part of the nanoparticles shows the hydrophilic functional group, and consequently other contrast material can be grown or bound. The intermolecular interaction includes a hydrophobic interaction, a hydrogen bond, a Van der Waals force, and so on.

Another example of the preferable organic separating layer according to the present invention is a lipid. Lipid may be effectively used in the separating layer due to an amphiphilic ligand containing both a hydrophobic and a hydrophilic region.

The preferable example of the separating layer in the present invention may be a multi-component hybrid structure consisting of the above-mentioned organic material and inorganic material.

According to a preferable embodiment, the separating layer includes:

(i) a metal chalcogen, MₓA_{y} [M=one or more elements selected from the group consisting of Group 2 metal elements (Be, Mg, Ca, Sr, Ba), Group 13 metal elements (Al, In, Tl), Group 14 metal elements (Si, Ge, Sn, Pb), Group 15 metal elements (As, Sb, Bi), transition metal elements (Sc, Ti, V, Zn, Y, Zr, Nb, Mo), Lanthanide metal elements (Ce, Pr, Nd, Pm, Sm, Eu, Lu) and Actinide metal elements; A is one or more elements selected from the group consisting of O, S, Se and Te; 0<x≦16, 0<y≦8], or a multi-component hybrid structure thereof;

(ii) a ceramic material [titania, zirconia, silica, alumina, aluminate-containing inorganic compound, silicate-containing inorganic compound, zeolite, titanate-containing inorganic compound, ZnO, calcite, apetite-containing inorganic compound, Sialon (silicon aluminium oxynitride), borate-containing inorganic compound, tungstate-containing inorganic compound, molybdate-containing inorganic compound, selenide-containing inorganic compound, telluride-containing inorganic compound, tantalate-containing inorganic compound, cuprite (Cu₂O), Ceria, a porous material (example: MCM (mesoporous crystalline material)-41, MCM-48, SBA-15, SBA-6 a mesoporous or microporous material), or a multi-component hybrid structure thereof];

(iii) a Polymer; (iv) a protein; (v) a carbohydrate; (vi) an amphiphilic surfactant; or (vii) a multi-component hybrid structure thereof.

In the T1-T2 dual modal MRI contrast agent of the present invention, T1 contrast material may be a material having various forms. Preferably, the T1 contrast material grows on the separating layer, or is bound to the separating layer by a covalent bond, a coordination bond, an ionic bond, a hydrogen bond, a hydrophilic interaction, a hydrophobic interaction or a Van der Waals force. In addition, T1 contrast materials may be included in the separating layer capable of accommodating a contrast material.

For example, the T1 contrast material grows on the separating layer, or is bound to the separating layer in the form of a chelate compound. In attachment as the chelate compound, a chelating ligand is attached to the surface of the separating layer and then a metal ion is bound via a coordination bond, and the resulting metal-chelating compound may be bound to the surface of the separating layer through a covalent bond.

According to a preferable embodiment, the T1 contrast material capable of being used in the present invention includes any one of materials generating a T1 signal. More preferably, the T1 contrast material capable of being used in the present invention includes a magnetic material, and much more preferably paramagnetic metal containing material.

Preferably, the T1 contrast material of the present invention includes a metal, an ion, a metal compound, a metal complex compound or a multi-component hybrid structure thereof.

The metal component capable of being used as the T1 contrast material in the present invention is a magnetic metal having an unpaired electron, and more preferably is selected from the group consisting of transition metal elements, Lanthanide metal elements and Actinide metal elements, and most preferably is one or more elements selected from the group consisting of Lanthanide metal elements Ce, Pr, Nd, Pm, Sm, Gd, Eu, Tb, Dy, Ho, Er, Tm, Yb, Lu) transition metal elements (Ti, V, Cr, Mn, Fe, Co, Ni, Cu, Ru) and a multi-component hybrid structure thereof.

The ion capable of being used as the T1 contrast material in the present invention is selected from all kinds of single atomic or polyatomic ions containing an unpaired electron, more preferably one or more Mⁿ⁺ (M=element selected from the group consisting of transition metal elements, Lanthanide metal elements and Actinide metal elements; 0<n≦14), and most preferably one or more single atomic or polyatomic ions selected from the group consisting of Tiⁿ⁺, Vⁿ⁺, Crⁿ⁺, Mnⁿ⁺, Feⁿ⁺, Coⁿ⁺, Niⁿ⁺, Cuⁿ⁺, Ruⁿ⁺, Gdⁿ⁺, Dyⁿ⁺, Hoⁿ⁺, Tbⁿ⁺, Tmⁿ⁺ and Ybⁿ⁺ (0<n≦14).

The metal compound capable of being used as the T1 contrast material in the present invention includes a metal chalcogen (Group 16 element) compound, a metal pnicogen (Group 15 element) compound, a metal carbon (Group 14 element) compound, a metal boron (Group 13 element) compound or a multi-component hybrid structure thereof.

The metal chalcogen compound capable of being used as the T1 contrast material in the present invention includes M^{a}ₓA_{z}, M^{a}ₓM^{b}_{y}A_{z} (M^{a}=one or more elements selected from the group consisting of transition metal elements, Lanthanide metal elements and Actinide metal elements; M^{b}=one or more elements selected from the group consisting of Group 1 metal elements, Group 2 metal elements, Group 13-15 elements, Group 17 elements, transition metal elements, Lanthanide metal elements and Actinide metal elements; A is one or more elements selected from the group consisting of O, S, Se, Te and Po; 0<x≦16, 0≦y≦16, 0<z≦8), or the multi-component hybrid structure thereof. According to a preferable embodiment, T1 contrast material used in the present invention is M^{a}ₓA_{z} or M^{a}ₓM^{b}_{y}A_{z} [M^{a}=one or more elements selected from the group consisting of Lanthanide metal elements (Ce, Pr, Nd, Pm, Sm, Gd, Eu, Tb, Dy, Ho, Er, Tm Yb, Lu) and transition metal elements (Ti, V, Cr, Mn, Fe, Co, Ni, Cu, Ru); M^{b}=one or more elements selected from the group consisting of Group 1 metal elements, Group 2 metal elements, Group 13-15 elements, Group 17 elements, transition metal elements, Lanthanide metal elements and Actinide metal elements; A is one or more elements selected from the group consisting, of O, S, Se, Te and Po; 0<x≦16, 0≦y≦16, 0<z≦8], or a multi-component hybrid structure thereof, and most preferably M^{a}ₓOₓ, M^{a}ₓM^{b}_{y}O_{z} [M^{a} one or more elements selected from the group consisting of Lanthanide metal elements (Ce, Pr, Nd, Pm, Sm, Gd, Eu, Tb, Dy, Ho, Er, Tm, Yb, Lu) and transition metal elements (Ti, V, Cr, Mn, Fe, Co, Ni, Cu, Ru); M^{b}=one or more elements selected from the group consisting of Group 1 metal elements, Group 2 metal elements, Group 13-15 elements, Group 17 elements, transition metal elements, Lanthanide metal elements and Actinide metal elements; 0<x≦16, 0≦y≦16, 0<z≦8], or a multi-component hybrid structure thereof.

The metal pnicogen compound capable of being used as the T1 contrast material in the present invention includes M^{c}ₓA_{z}, M^{c}ₓM^{d}_{y}A_{z} (M^{c}=one or more elements selected from the group consisting of transition metal elements, Lanthanide metal elements and Actinide metal elements; M^{d}=one or more elements selected from the group consisting of Group 1 metal elements, Group 2 metal elements, Group 13 elements, Group 14 elements, Group 16 elements, Group 17 elements, transition metal elements, Lanthanide metal elements and Actinide metal elements; A is one or more elements selected from the group consisting of N, P, As, Sb and Bi; 0<x≦24, 0≦y≦24, 0<z≦8), or a multi-component hybrid structure thereof, and most preferably M^{c}ₓA_{z}, M^{c}ₓM^{d}_{y}A_{z} [M^{c}=one or more elements selected from the group consisting of Lanthanide metal elements (Ce, Pr, Nd, Pm, Sm, Gd, Eu, Tb, Dy, Ho, Er, Tm, Yb, Lu) and transition metal elements (Ti, V, Cr, Mn, Fe, Co, Ni, Cu, Ru); M^{d}=one or more elements selected from the group consisting of Group 1 metal elements, Group 2 metal elements, Group 13 elements, Group 14 elements, Group 16 elements, Group 17 elements, transition metal elements, Lanthanide metal elements and Actinide metal elements; A is one or more elements selected from the group consisting of N, P, As, Sb and Bi; 0<x≦24, 0≦y≦24, 0<z≦8], or a multi-component hybrid structure thereof.

The metal carbon compound capable of being used as the T1 contrast material in the present invention includes M^{e}ₓA_{z}, M^{e}ₓM^{f}_{y}A_{z} (M^{e} one or more elements selected from the group consisting of transition metal elements, Lanthanide metal elements and Actinide metal elements; M^{f}=one or more elements selected from the group consisting of Group 1 metal elements, Group 2 metal elements, Group 13 elements, Group 15-17 elements, transition metal elements, Lanthanide metal elements and Actinide metal elements; A is one or more elements selected from the group consisting of C, Si, Ge, Sn and Pb; 0<x≦32, 0≦y≦32, 0<z≦8), or a multi-component hybrid structure thereof, and most preferably M^{e}ₓA_{z} M^{e}ₓM^{f}_{y}A_{z} [M^{e}=one or more elements selected from the group consisting of Lanthanide metal elements (Ce, Pr, Nd, Pm, Sm, Gd, Eu, Tb, Dy, Ho, Er, Tm, Yb, Lu) and transition metal elements (Ti, V, Cr, Mn, Fe, Co, Ni, Cu, Ru); M^{f}=one or more elements selected from the group consisting of Group 1 metal elements, Group 2 metal elements, Group 13 elements, Group 15-17 elements, transition metal elements, Lanthanide metal elements and Actinide metal elements; A is one or more elements selected from the group consisting of C, Si, Ge, Sn and Pb; 0<x≦32, 0≦y≦32, 0<z≦8], or a multi-component hybrid structure thereof.

The metal boron compound capable of being used as the T1 contrast material in the present invention includes M^{g}A_{z}, M^{g}ₓM^{h}_{y}A_{z} (M^{g}=one or more elements selected from the group consisting of transition metal elements, Lanthanide metal elements and Actinide metal elements; M^{h}=one or more elements selected from the group consisting of Group 1 metal elements, Group 2 metal elements, Group 14-17 elements, transition metal elements, Lanthanide metal elements and Actinide metal elements; A is one or more elements selected from the group consisting of B, Al, Ga, In and Tl; 0<x≦40, 0≦y≦40, 0<z≦8), or a multi-component hybrid structure thereof, and most preferably MⁱₓA_{z}, MⁱₓM^{j}_{y}A_{z} [Mⁱ=one or more elements selected from the group consisting of Lanthanide metal elements (Ce, Pr, Nd, Pm, Sm, Gd, Eu, Tb, Dy, Ho, Er, Tm, Yb, Lu) and transition metal elements (Ti, V, Cr, Mn, Fe, Co, Ni, Cu, Ru); M^{j} one or more elements selected from the group consisting of Group 1 metal elements, Group 2 metal elements, Group 14-17 elements, transition metal elements, Lanthanide metal elements and Actinide metal elements; A is one or more elements selected from the group consisting of B, Al, Ga, In and Tl; 0<x≦40, 0≦y≦40, 0<z≦8), or a multi-component hybrid structure thereof.

Still another example of the T1 contrast material in the present invention is a metal complex compound. The metal complex compound refers all materials consisting of a center metal and a ligand bound to the metal coordinately, and particularly a center metal having T1 effect or a complex compound composed of a center metal and a coordination ligand. Preferably, the metal complex compound capable of being used as the T1 contrast material in the present invention includes M^{k}ₓL_{z}, M^{k}ₓM^{l}_{y}L_{z} (M^{k}=one or more elements selected from the group consisting of transition metal elements, Lanthanide metal elements and Actinide metal elements; M^{l}=one or more elements selected from the group consisting of Group 1 metal elements, Group 2 metal elements, Group 13-17 elements, transition metal elements, Lanthanide metal elements and Actinide metal elements; L=one or more ligands capable of binding with a metal through a coordination bond; 0<x≦10, 0<y≦10, 0<z≦240), or a multi-component hybrid structure thereof. More preferably, the metal complex compound capable of being used as the T1 contrast material in the (Mk present invention includes M^{k}ₓL_{z}, M^{k}ₓM^{l}_{y}L_{z} (M^{k}=one or more elements selected from the group consisting of transition metal elements, Lanthanide metal elements and Actinide metal elements; M^{l}=one or more elements selected from the group consisting of Group 1 metal elements, Group 2 metal elements Group 13 elements, Group 14 elements, Group 16 elements, transition metal elements, Lanthanide metal elements and Actinide metal elements; L=one or more ligands with a functional group containing oxygen, sulfur, boron, nitrogen, selenium, tellurium or phosphorous; 0<x≦10, 0<y≦10, 0<z≦240), or a multi-component hybrid structure thereof.

The ligand of the metal complex compound includes, but not limited to, a ligand having one or more functional groups selected from the group consisting of —(C═O)—, —COOH, —NH₂, —SH, —CONH₂, —PO₃H, —OPO₄H₂, —SO₃H, —OSO₃H, —NO₂, —CHO, —COSH, —CN, —N₃, —N₂, —OH, —SCOCH₃, —SCN, —NCS, —NCO, —OCN, —N—, —NH—, —S—, —O—, —Se—, —Te—, —NO₃, —CO₃, —CONHR, —CONR— and NR¹R²(R (R¹ and R²)═CₙHₘ, 0≦n≦16, 0≦m≦34).

The ligand of the metal complex compound includes a chelating ligand which is simultaneously attached to a central metal ion by bonds from two or more functional groups. Preferably, the chelating ligand includes, but not limited to, EDTA (ethylenediaminotetracetic acid), DTPA (diethylenetriaminopentaacetic acid), EOB-DTPA (N-[2-[bis(carboxymethyl)amino]-3-(4-ethoxyphenyl)propyl]-N-[2-[bis(carboxymethyl)amino]ethyl]-L-glycine), DTPA-GLU (N,N-bis[2-[bis(carboxymethyl)amino]ethyl]-L-glutamic acid), DTPA-LYS (N,N-bis[2-[bis(carboxymethyl)amino]ethyl]-L-lysine), DTPA-BMA (N,N-bis[2-[carboxymethyl[(methylcarbamoyl)methyl]amino]ethyl]glycine), BOPTA (4-carboxy-5,8,11-tris(carboxymethyl)-1-phenyl-2-oxa-5,8,11-triazamidecan-13-oic acid), DOTA (1,4,7,10-tetraazacyclodoclecan-1,4,7,10-tetraacetic acid), DO3A (1,4,7,10-tetraazacyclododecan-1,4,7-triacetic acid), HPDO3A (10-(2-hydroxypropyl)-1,4,7,10-tetraazacyclododecan-1,4,7-triacetic acid) MCTA (2-methyl-1,4,7,10-tetraazacyclododecane-1,4,7,10-tetraacetic acid), DOTMA ((α,α′,α″,α′″)-tetramethyl-1,4,7,10-tetraazacyclododecan-1,4,7,10-tetraacetic acid), PCTA (3,6,9,15-tetraazabicyclo[9.3.1]pentadeca-1(15),11,13-triene-3,6,9-triacetic acid), BOPTA (4-carboxy-5,8,11-bis(carboxymethyl)-1-phenyl-12-(phenylmethoxy)methyl-8-phosphomethyl-2-oxa-5,8,11-triazamidecan-12-oid acid), N,N′-[(phosphomethylimino)di-2,1-ethanediyl]bis[N-(carboxymethyl)glycine] (N,N′-phosphonomethylimino-di-2,1-ethanediyl-bis(N-carboxymethylglycine)), N,N′-[(phosphomethylimino)di-2,1-ethanediyl]bis[N-(carboxymethyl)glycine] (N,N′-phosphonomethylimino-di-2,1-ethanediyl-bis(n-phosphonomethyl)glycine)), N,N′-[(phosphinomethylimino)di-2,1-ethanediyl]bis[N-(carboxymethyl)glycine] (N,N′-(phosphinomethylimino-di-2,1-ethanediyl-bis-(N-(carboxymethyl)glycine), DOTP (1,4,7,10-tetraazacyclodecane-1,4,7,10-tetrakis(methylphosphonic acid), DOTMP (1,4,7,10-tetraazacyclodecane-1,4,7,10-tetrakis methylene(methylphosphinic acid), or a derivative thereof.

The preferable example of the T1 contrast material in the present invention is the multi-component hybrid structure of an ion, a metal, a metal compound or a metal complex compound capable of being used in the T1 contrast agent as described above. As a preferable example of the multi-component hybrid structure, the multi-component hybrid structure includes, but not limited to, a compound in which the inorganic compound is further coordinated to the metal complex compound, or the ligand is substituted for an element consisting of the inorganic compound.

As illustrative example of the multi-component hybrid structure, Ln₂O(CO₃)₂ (Ln=one or more elements selected from the group consisting of Gd, Dy, Er, Ho, Tb and Yb) may be used as the T1 contrast agent in the present invention, and is the multi-component hybrid structure which substitutes CO₃²⁻ ligand for two oxygen atoms of Ln₂O₃ (Ln=Gd, Dy, Er, Tb, Ho) belonging to a metal oxide.

In addition, the multi-component hybrid structure capable of being used as the T1 contrast agent in the present invention may be present in various structures and forms using a mixture of an ion, a metal, a metal compound or a metal complex compound.

According to a more preferable embodiment, the T1 contrast material includes:

(i) a metal ion, Mⁿ⁺ (M=Gd, Tb, Dy, Ho, Er, Mn),

(ii) a metal chalcogen, MₓO_{y} (M=one or more elements selected from the group consisting of Gd, Tb, Dy, Ho, Er and Mn; 0<x≦16, 0≦y≦8),

(iii) a metal-chelating compound,

(iv) a multi-component hybrid structure of the metal chalcogen and the metal complex compound, or

(v) a multi-component hybrid structure thereof.

In the T1-T2 dual modal MRI contrast agent of the present invention, the T2 contrast material may be a variety of structures and forms. Preferably, the T2 contrast material is present in a core and grows on the separating layer, or is bound to the separating layer by a hydrogen bond, a hydrophilic interaction, a hydrophobic interaction or a Van der Waals force. In addition, the T2 contrast material may be involved in the center of the separating layer capable of accommodating a contrast material to sufficiently keep a distance to the T1 contrast material.

The T2 contrast material used in the present invention may include any material generating a T2 signal. The T2 contrast material capable of being used in the present invention includes a magnetic material, and preferably a ferromagnetic, ferrimagnetic or supermagnetic material.

According to a preferable embodiment, the T2 contrast material is a metal, a metal compound, an alloy or a multi-component hybrid structure thereof.

The metal capable of being used as the T2 contrast material in the present invention includes transition metal elements, Lanthanide metal elements, Actinide metal elements or the multi-component hybrid structure thereof. More preferably, the metal is Co, Fe, Ni or a multi-component hybrid structure thereof.

The metal capable of being used as the T2 contrast material in the present invention includes a metal chalcogen (Group 16 element) compound, a metal pnicogen (Group 15 element) compound, a metal carbon (Group 14 element) compound, a metal boron (Group 13 element) compound or a multi-component hybrid structure thereof.

The metal chalcogen compound capable of being used as the T2 contrast material in the present invention includes M^{a}ₓA_{z}, M^{a}ₓM^{b}_{y}A_{z} (M^{a}=one or more elements selected from the group consisting of transition metal elements, Lanthanide metal elements and Actinide metal elements; M^{b}=one or more elements selected from the group consisting of Group 1 metal elements, Group 2 metal elements, Group 13-15 elements, Group 17 elements, transition metal elements, Lanthanide metal elements and Actinide metal elements; A is one or more elements selected from the group consisting of O, S, Se, Te and Po; 0<x≦16, 0≦y≦16, 0<z≦8), or a multi-component hybrid structure thereof.

More preferably, the metal chalcogen compound used in the present invention is M^{a}ₓA_{z}, M^{a}ₓM^{b}_{y}A_{z} (M^{a}=one or more transition metal elements selected from the group consisting of Cr, Mn, Fe, Co, Ni, Cu and Zn; M^{b}=one or more elements selected from the group consisting of Group 1 metal elements, Group 2 metal elements, transition metal elements, Group 13-15 elements, Group 17 elements, Lanthanide metal elements and Actinide metal elements; A is one or more elements selected from the group consisting of O, S, Se, Te and Po; 0<x≦16, 0≦y≦16, 0<z≦8), or a multi-component hybrid structure thereof.

Much more preferably, the metal chalcogen compound used in the present invention is M^{a}ₓO_{z}, M^{a}ₓM^{b}O_{z} (M^{a}=one or more transition metal elements selected from the group consisting of Cr, Mn, Fe, Co, Ni, Cu and Zn; M^{b}=one or more elements selected from the group consisting of Group 1 metal elements, Group 2 metal elements, transition metal elements, Group 13-15 elements, Group 17 elements, Lanthanide metal elements and Actinide metal elements; 0<x≦16, 0≦y≦16, 0≦z≦8), or a multi-component hybrid structure thereof.

The metal pnicogen compound capable of being used as the T2 contrast material in the present invention includes M^{c}ₓA_{z}, M^{c}ₓM^{d}_{y}A_{z} (M^{c}=one or more elements selected from the group consisting of transition metal elements, Lanthanide metal elements and Actinide metal elements; M^{d}=one or more elements selected from the group consisting of Group 1 metal elements, Group 2 metal elements, Group 13-14 elements, Group 16-17 elements, transition metal elements, Lanthanide metal elements and Actinide metal elements; A is one or more elements selected from the group consisting of N, P, As, Sb and Bi; 0<x≦24, 0≦y≦24, 0<z≦8), or a multi-component hybrid structure thereof.

More preferably, the metal pnicogen compound includes M^{c}ₓA_{z}, M^{c}ₓM^{d}_{y}A_{z} (M^{c}=one or more transition metal elements selected from the group consisting of Cr, Mn, Fe, Co, Ni, Cu and Zn; M^{d}=one or more elements selected from the group consisting of Group 1 metal elements, Group 2 metal elements, transition metal elements, Group 13-14 elements, Group 16-17 elements, Lanthanide metal elements and Actinide metal elements; A is one or more elements selected from the group consisting of N, P, As, Sb and Bi; 0<x≦24, 0≦y≦24, 0<z≦8), or a multi-component hybrid structure thereof.

The metal carbon compound capable of being used as the T2 contrast material in the present invention includes M^{e}ₓA_{z}, M^{e}ₓM^{f}_{y}A_{z} (M^{e}=one or more elements selected from the group consisting of transition metal elements, Lanthanide metal elements and Actinide metal elements; M^{f}=one or more elements selected from the group consisting of Group 1 metal elements, Group 2 metal elements, Group 13 elements, Group 15-17 elements, transition metal elements, Lanthanide metal elements and Actinide metal elements; A is one or more elements selected from the group consisting of C, Si, Ge, Sn and Pb; 0<x≦32, 0≦y≦32, 0<z≦8), or a multi-component hybrid structure thereof, and most preferably M^{e}ₓA_{z}, M^{e}ₓM^{f}A_{z} [M^{e}=one or more transition metal elements selected from the group consisting of Cr, Mn, Fe, Co, Ni, Cu and Zn; M^{f}=one or more elements selected from the group consisting of Group 1 metal elements, Group 2 metal elements, Group 13 elements, Group 15-17 elements, transition metal elements, Lanthanide metal elements and Actinide metal elements; A is one or more elements selected from the group consisting of C, Si, Ge, Sn and Pb; 0<x≦32, 0y≦32, 0<z≦8], or a multi-component hybrid structure thereof.

The metal boron compound capable of being used as the T2 contrast material in the present invention includes M^{g}ₓA_{z}, M^{g}ₓM^{h}_{y}A_{z} (M^{g}=one or more elements selected from the group consisting of transition metal elements, Lanthanide metal elements and Actinide metal elements; M^{h}=one or more elements selected from the group consisting of Group 1 metal elements, Group 2 metal elements, Group 14-17 elements, transition metal elements, Lanthanide metal elements and Actinide metal elements; A is one or more elements selected from the group consisting of B, Al, Ga, In and Tl; 0<x≦40, 0≦y≦40, 0<z≦8), or a multi-component hybrid structure thereof, and most preferably M^{g}ₓA_{z}, M^{g}ₓM^{h}_{y}A_{z} [M^{g}=one or more transition metal elements selected from the group consisting of Cr, Mn, Fe, Co, Ni, Cu and Zn; M^{j}=one or more elements selected from the group consisting of Group 1 metal elements, Group 2 metal elements, Group 14-17 elements, transition metal elements, Lanthanide metal elements and Actinide metal elements; A is one or more elements selected from the group consisting of B, Al, Ga, In and Tl; 0<x≦40, 0≦y≦40, 0<z≦8), or a multi-component hybrid structure thereof.

Preferably, the alloy used as the T2 contrast material in the present invention includes M^{e}ₓM^{f}_{y}, M^{e}ₓM^{f}_{y}M^{g}_{z} (M^{e}=one or more elements selected from the group consisting of transition metal elements, Lanthanide metal elements and Actinide metal elements; M^{f} and M^{g}=one or more elements selected from the group consisting of Group 1 metal elements, Group 2 metal elements, Group 13 elements, Group 14 elements, Group 15 elements, Group 16 elements, Group 17 elements, transition metal elements, Lanthanide metal elements and Actinide metal elements; 0<x≦20, 0<y≦20, 0<z≦20), or a multi-component hybrid structure thereof, and more preferably one or more elements selected from the group consisting of transition metal elements selected from the group consisting of Ba, Cr, Mn, Fe, Co, Ni, Cu, Zn, Nb, Mo, Zr, Te, W, Pd, Ag, Pt and Au, Lanthanide metal elements selected from the group consisting of Gd, Tb, Dy, Ho, Er, Sm and Nd, and Actinide metal elements; M^{f} and M^{g}=one or more elements selected from the group consisting of Group 1 metal elements, Group 2 metal elements, Group 13 elements, Group 14 elements, Group 15 elements, Group 16 elements, transition metal elements, Lanthanide metal elements and Actinide metal elements; 0<x≦20, 0<y≦20, 0<z≦20), or a multi-component hybrid structure thereof.

The preferable example of the T2 contrast material used in the present invention is the multi-component hybrid structure of a metal, a metal compound or a metal alloy capable of being used in the T2 contrast agent as described above.

The multi-component hybrid structure capable of being used as the T2 contrast agent in the present invention has various structures and forms.

According to a more preferable embodiment, the T2 contrast material includes:

(i) a metal, M (M=Fe, Co, Ni),

(ii) a metal chalcogen, MₓFe_{y}O_{z} (M==one or more elements selected from transition metal elements containing Zn, Mn, Fe, Co or Ni; 0<x≦8, 0<y≦8, 0<z≦8), ZnₓFe_{y}O_{z}(0<x≦8, 0<y≦8, 0<z≦8), Zn_{w}MₓFe_{y}O_{z} (M represents one or more elements selected from transition metal elements containing Mn, Fe, Co or Ni; 0≦w≦8, 0≦x≦8, 0<y≦8, 0<z≦8),

(iii) a metal alloy, M^{e}ₓM^{f}_{y}, M^{e}ₓM^{f}_{y}M^{g}_{z} (M^{e}, M^{f} and M^{g} independently represents one or more elements selected from the group consisting of Co, Fe, Mn, Ni, Mo, Al, Cu, Pt, Sm, B, Bi, Cu, Sn, Sb, Ga, Ge, Pd, In, Au, Ag and Y), or

(iv) a multi-component hybrid structure thereof.

Preferably, the T1-T2 dual modal MRI contrast agent of the present invention has a size in a range of a nanometer or a micrometer, more preferably 1 nm-500 mm, and much more preferably 1-1000 nm.

The T1-T2 dual modal MRI contrast agent developed according to the present invention exhibits the solubility per se. In addition, the T1-T2 dual modal MRI contrast agent of the present invention may be coated with a water-soluble multi-functional ligand depending on the necessity. The present inventors have developed a novel technology for coating the surface of a nanoparticle (See, Korean Pat. Nos. 0652251, 0604976 and 0713745). The surface coating technology allows the particle of the present invention to enhance the solubility in water and to reduce cytotoxicity, enabling to be extensively applied into a biological diagnosis and treatment. In addition, the surface coating technology plausibly permits to introduction of other active ingredients.

According to a preferable embodiment, the water-soluble multi-functional ligand includes (i) an attachment region (L_{I}) to be linked to the surface of the shell, and more preferably (ii) an active ingredient-binding region (L_{II}) for bonding of active ingredients, or (iii) a cross-linking region (L_{III}) for cross-linking between water-soluble multi-functional ligands, or (iv) a region which includes both the active ingredient-binding region (L_{II}) and the cross-linking region (L_{III})

The term “attachment region (L_{I})” refers to a portion of the water-soluble multi-functional ligand including a functional group capable of binding to the nanoparticle, and preferably to an end portion of the functional group. Accordingly, it is preferable that the attachment region including the functional group should have high affinity with the materials constituting the nanoparticle. According to a preferable embodiment, the nanoparticle can be attached to the attachment region by an ionic bond, a covalent bond, a hydrogen bond, a Van der Waals force, a hydrophobic interaction or a coordination bond.

The term “active ingredient-binding region (L_{II})” means a portion of water-soluble multi-functional ligand containing the functional group capable of binding to chemical or biological functional substances, and preferably the other end portion located at the opposite side from the attachment region. The functional group of the active ingredient-binding region may be varied depending on the type of active ingredient and their formulae. The active ingredient-binding region in this invention includes, but not limited to, —SH, —CHO, —COOH, —NH₂, —OH, —PO₃H, —OPO₄H₂, —SO₃H, —OSO₃H, —NR₃⁺X⁻ (R═CₙHₘ, 0≦n≦16, 0≦m≦34, X═OH, Cl or Br), NR₄⁺X⁻ (R═CₙHₘ, 0≦n≦16, 0≦m≦34, X═OH, Cl or Br), —N₃, —SCOCH₃, —SCN, —NCS, —NCO, —CN, —F, —Cl, —I, —Br, an epoxy group, —ONO₂, —PO(OH)₂, —C═NNH₂, —HC═CH— and —C≡C—.

The term “cross-linking region (L_{III})” refers to a portion of the multi-functional ligand including the functional group capable of cross-linking to an adjacent water-soluble multi-functional ligand, and preferably a side chain attached to a central portion. The term “cross-linking” means that the multi-functional ligand is bound to another multi-functional ligand by intermolecular interaction. The intermolecular interaction includes, but not particularly limited to, a hydrogen bond, a covalent bond (e.g., disulfide bond), an ionic bond, and so on. Therefore, the cross-linkable functional group may be variously selected according to the kind of the intermolecular interaction of interest. For example, the cross-linking region may include —SH, —CHO, —COOH, —NH₂, —OH, —PO₃H, —OPO₄H₂, —SO₃H, —OSO₃H, —NR₃⁺X⁻ (R═CₙHₘ, 0≦n≦16, 0≦m≦34, X═OH, Cl or Br), NR₄⁺X⁻ (R═CₙHₘ, 0≦n≦16, 0≦m≦34, X═OH, Cl or Br), —N₃, —SCOCH₃, —SCN, —NCS, —NCO, —CN, —F, —Cl, —I, —Br, an epoxy group, —ONO₂, —PO(OH)₂, —C═NNH₂, —C═C— and —C═C— as the functional ligand, but not limited to.

According to a preferable embodiment, the water-soluble multi-functional ligand of the present invention includes a biocompatible polymer, a peptide, a protein, an amphiphilic ligand, a nucleic acid and a lipid.

The method to obtain MR imaging by the T1-T2 dual modal contrast agent of the present invention may be carried out according to a conventional method and device. MR imaging methods and devices are disclosed in D. M. Kean and M. A. Smith, Magnetic Resonance Imaging: Principles and Applications (William and Wilkins, Baltimore 1986), U.S. Pat. Nos. 6,151,377, 6,144,202, 6,128,522, 6,127,825, 6,121,775, 6,119,032, 6,115,446, 6,111,410 and 602,891, which are incorporated herein by reference.

The T1-T2 dual-modal contrast agent of the present invention may generate both T1 and T2 signal and thus observe the signal complementarily, resulting in accurate diagnosis through reduction of misdiagnosis. In comparison with other multi-modal imaging methods, the T1-T2 dual-modal contrast agent of the present invention may remarkably reduce a diagnosis cost due via simple operation within the same MR imaging device, and obtain both T1 and T2 MR imaging by one administration of contrast agent and simple manipulation of MR device.

The T1-T2 dual-modal MRI contrast agent of the present invention is primarily used in MR imaging and further may be used in multi-modal contrast according to combination with a material permitting other type of imaging. Other type of contrast material may be directly bound to the contrast agent, or indirectly linked to the contrast agent through the multi-functional ligand coated on the contrast agent, or constituted with a carrier.

The T1-T2 dual-modal MRI contrast agent of the present invention may be used in SPECT (Single Photon Emission Computed Tomography) or PET (Positron Emission Tomography) by combination with a radioisotope. The preferable example of radioisotope useful in the present invention includes ¹⁰C, ¹¹C, ¹³O, ¹⁴O, ¹⁵O, ¹²N, ¹³N, ¹⁵F, ¹⁷F, ³²C, ³³Cl, ³⁴C, ⁴³Sc, ⁴⁴Sc, ⁴⁵Ti, ⁵¹Mn, ⁵²Mn, ⁵²Fe, ⁵³Fe, ⁵⁵Co, ⁵⁶Co, ⁵⁸Co, ⁶¹Cu, ⁶²Cu, ⁶²Zn, ⁶³Zn, ⁶⁴Cu, ⁶⁵Zn, ⁶⁶Ga, ⁶⁶Ge, ⁶⁷Ge, ⁶⁸Ga, ⁶⁹Ge, ⁶⁹As, ⁷⁰As, ⁷⁰Se, ⁷¹Se, ⁷¹As, ⁷²As, ⁷³Se, ⁷⁴Kr, ⁷⁴Br, ⁷⁵Br, ⁷⁶Br, ⁷⁷Br, ⁷⁷Kr, ⁷⁸Br, ⁷⁸Rb, ⁷⁹Rb, ⁷⁹Kr, ⁸¹Rb, ⁸²Rb, ⁸⁴Rb, ⁸⁴Zr, ⁸⁵Y, ⁸⁶Y, ⁸⁷Y, ⁸⁷Zr, ⁸⁸Y, ⁸⁹Zr, ⁹²Tc, ⁹³Tc, ⁹⁴Tc, ⁹⁵Tc, ⁹⁵Ru, ⁹⁵Rh, ⁹⁶Rh, ⁹⁷Rh, ⁹⁸Rh, ⁹⁹Rh, ¹⁰⁰Rh, ¹⁰¹Ag, ¹⁰²Ag, ¹⁰²Rh, ¹⁰³Ag, ¹⁰⁴Ag, ¹⁰⁵Ag, ¹⁰⁶Ag, ¹⁰⁸In, ¹⁰⁹In, ¹¹⁰In, ¹¹⁵Sb, ¹¹⁶Sb, ¹¹⁷Sb, ¹¹⁵Te, ¹¹⁶Te, ¹¹⁷Te, ¹¹⁷I, ¹¹⁸I, ¹¹⁸Xe, ¹¹⁹Xe, ¹¹⁹I, ¹¹⁹Te, ¹²⁰I, ¹²⁰Xe, ¹²¹Xe, ¹²¹I, ¹²²I, ¹²³Xe, ¹²⁴I, ¹²⁶I, ¹²⁸I, ¹³¹I, ¹²⁹La, ¹³⁰La, ¹³¹La, ¹³²La, ¹³³La, ¹³⁵La, ¹³⁶La, ¹⁴⁰Sm, ¹⁴¹Sm, ¹⁴²Sm, ¹⁴⁴Gd, ¹⁴⁵Gd, ¹⁴⁵Eu, ¹⁴⁶Gd, ¹⁴⁶Eu, ¹⁴⁷Eu, ¹⁴⁷Gd, ¹⁴⁸Eu, ¹⁵⁰Eu, ¹⁹⁰Au, ¹⁹¹Au, ¹⁹²Au, ¹⁹³Au, ¹⁹³Tl, ¹⁹⁴Tl, ¹⁹⁴Au, ¹⁹⁵T, ¹⁹⁶Tl, ¹⁹⁷Tl, ¹⁹⁸Tl, ²⁰⁰Tl, ²⁰⁰Bi, ²⁰²Bi, ²⁰³Bi, ²⁰⁵Bi, ²⁰⁶Bi or a derivative thereof, but not limited to.

PET imaging methods and devices are disclosed in U.S. Pat. Nos. 6,151,377, 6,072,177, 5,900,636, 5,608,221, 5,532,489, 5,272,343 and No. 5,103,098, which are incorporated herein by reference. In addition, SPECT imaging method and devices are disclosed in U.S. Pat. Nos. 6,115,446, 6,072,177, 5,608,221, 5,600,145, 5,210,421 and 5,103,098, which are incorporated herein by reference.

Furthermore, the T1-T2 dual-modal MRI contrast agent of the present invention may be used in an optical imaging and spectroscopy in combination with the fluorescent substance. For obtaining an optical image, preferably a luminescent, fluorescent or chemiluminescent substance is directly bound to the dual-modal MRI contrast agent of the present invention, or is indirectly linked to the water-soluble multi-functional ligand.

The example of the above-described fluorescent substance includes, but not limited to, fluorescein, rhodamine, lucifer yellow, B-phytoerythrin, 9-acrydine isothiocyanate, lucifer yellow VS, 4-acetamido-4′-isothio-cyanatostilbene-2,2′-disulfonate, 7-diethylamino-3-(4′-isothiocyatophenyl)-4-methylcournarin, succinimidyl-pyrenebutyrate, 4-acetamido-4′-isothio-cyanatostilbene-2,2′-disulfonate derivatives, LC™-Red 640, LC™-Red 705, Cy5, Cy5.5, Alexa dye series, resamine, isothiocyanate, erythrin isothiocyanate, diethyltriamine pentaacetate, 1-dimethylaminonaphthyl-5-sulfonate, 1-anilino-8-naphthalene, 2-p-toluidinyl-6-naphthalene, 3-phenyl-7-isocyanatocournarin, 9-isothiocyanatoacridine, acridine orange, N-(p-(2-benzoxazolyl)phenyl)maleimide, benzoxadiazol, stilbene and pyrene and a derivative thereof, a silica nanoparticle containing a fluorescent substance, Group II/VI semiconductor quantum dot, Group III/V semiconductor quantum dot, Group VI semiconductor quantum dot, or the multi-component hybrid structure thereof. Further, the optical substance includes a gold nanoparticle, a silver nanoparticle or the multi-component hybrid structure thereof, but not limited to. General description of optical imaging is disclosed in U.S. Pat. No. 5,650,135, which is incorporated herein by reference.

In addition, the dual modal MRI nanoparticle of the present invention linked with various substance (example: barium sulfate, iodine a derivative containing iodine, or the multi-component hybrid structure thereof) having X-ray contrast effect may be used in CT imaging. For obtaining CT images, CT imaging may be carried out according to the methods'disclosed in U.S. Pat. No. 6,151,377, No. 5,946,371, No. 5,446,799, No. 5,406,479, No. 5,208,581 and No. 5,109,397, which are incorporated herein by reference.

As described above, the contrast agent of this invention may primarily permit to obtain T1 and T2 MR imaging, and also perform various imaging or different chemical/biological function (for example, cell chasing, cancer treatment) using further introduction of other functional material. A biomolecule (example: an antibody, a protein, an antigen, a peptide, a nucleic acid, an enzyme, a cell, etc.) or a bioactive chemical substance (example: a monomer, a polymer, an inorganic support, a drug, etc.) may be bound to a T1-T2 dual modal MRI contrast agent by a covalent bond, an ionic bond, a hydrophilic interaction, a Van der Waals force, an electrostatic interaction or a hydrophobic interaction. The linkage may be carried out using direct binding to the surface of the contrast agent, or indirectly binding to the contrast agent through the multi-functional ligand coated on the contrast agent. In addition, the carrier in which a dual modal contrast agent and an active substance are involved together may be used.

The example of additional biomolecules includes an antibody, a protein, an antigen, a peptide, a nucleic acid, an enzyme, a cell and so on. Preferably, the example of biomolecule includes a protein, a peptide, DNA, RNA, an antigen, hapten, avidin, streptavidin, neutravidin, protein A, protein G, lectin, selectin, a hormone, an interleukin, an interferon, a growth factor, a tumor necrosis factor, endotoxin, lymphotoxin, urokinase, streptokinase, tissue plasminogen activator, hydrolase, oxido-reductase, lyase, biological active enzymes such as isomerase and synthetase, enzyme cofactor and enzyme inhibitor, or a derivative thereof, but not limited to.

The chemical active substance includes several functional monomers, polymers, inorganic substances, or drugs.

Exemplified monomer described above includes, but not limited to, a drug containing anti-cancer drug, antibiotics, Vitamin and folic acid, a fatty acid, a steroid, a hormone, a purine, a pyrimidine, a monosaccharide and a disaccharide.

The example of the above-described bioactive chemical polymer includes dextran, carbodextran, polysaccharide, cyclodextran, pullulan, cellulose, starch, glycogen, carbohydrate, monosaccharides, disaccharides and oligosaccharides, polyphosphagen, polylactide, polylactide-co-glycolide, polycaprolactone, polyanhydride, polymaleic acid and a derivative of polymaleic acid, polyalkylcyanoacrylate, polyhydroxybutylate, polycarbonate, polyorthoester, polyethylene glycol, poly-L-lysine, polyglycolide, polymethyl methacrylate, polymethylether methacrylate, polyvinylpyrrolidone, or a derivative thereof, but not limited to.

The illustrative example of the above-described bioactive inorganic substance includes, but not limited to, a metal chalcogen compound, an inorganic ceramic material, a carbon material, a semiconductor substrate consisting of Group II/VI elements, Group III/VI elements and Group IV elements, a metal or complex of metal, and preferably, SiO₂, TiO₂, zirconia, a porous material, indium tin oxide (ITO), nanotube, graphite, fullerene, CdS, CdSe, CdTe, ZnO, ZnS, ZnSe, ZnTe, Si, GaAs, AlAs, Au, Pt, Ag, Cu, and so forth.

In another aspect of this invention, the present invention provides a heat-generating composition containing a particle with the above-described core-separating layer-shell structure.

Based on the previous patent application of the present inventors, we have disclosed a heat generation ability of a magnetic nanoparticle (Korean Pat. Appln. Nos. 2008-004659 and 2008-0046589). A hyperthermia using heat released from a magnetic nanoparticle has been discussed in many references (R. E. Rosensweig, J. Magn. Magn. Mater. 2002, 252, 370; US Pat. Appln. Pub. No. 20050090732; U.S. Pat. No. 6,541,039; WO 2006/102307; and U.S. Pat. No. 7,282,479). Further, a nanoparticle consisting of a first layer-separating layer-second layer structure includes a magnetic material, and it is well-known to those ordinarily skilled in the art that the particle of the present invention may be used as a heat-generating composition because the magnetic property of the particle is almost equal in comparison with that of each component per se.

Since the material of the present invention has very remarked heat-generation coefficient, it may be used not only in a variety of heat-generating devices but also in hyperthermia or drug release for biomedical purpose. In more detail the heat-generating composition of the present invention may be applied to uses such as cancer treatment, pain relief, vessel treatment, bone recovery, drug activation or drug release. In particular, the heat-generating composition of the present invention has a utility as a composition for hyperthermia.

In still another aspect of this invention, the present invention provides a drug delivery system containing a particle with the above-described first layer-separating layer-second layer structure.

The fact that a nanoparticle possesses the utility as drug delivery carrier has been disclosed in many references (Roco, M. C. Nanotechnology: Convergence with modern biology and medicine. Curr. Opin. Biotechnol. 2003, 14, 337.). Furthermore, the present inventors have demonstrated that a nanoparticle may effectively penetrate a blood-brain barrier (BBB) so as to deliver a drug (Korean Pat. Appln. No. 2008-0043666). Therefore, the first layer-separating layer-second layer particle of this invention has a utility in a drug delivery system. In addition, the drug delivery effect through physiochemical stimulation may be controlled using a heat generation ability of nanoparticles.

The T1-T2 dual modal MRI contrast agent, heat-generating composition and drug delivery composition of the present invention may be administrated together with a pharmaceutically acceptable carrier, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate and mineral oils. Details of suitable pharmaceutically acceptable carriers and formulations can be found in Remington's Pharmaceutical Sciences (19th ed., 1995), which is incorporated herein by reference.

Preferably, the contrast agent, heat-generating composition and drug delivery composition of the present invention may be parenterally administered. In the case that the contrast agent is administered parenterally, it is preferably administered by intravenous, intramuscular, intralesional or intracranial injection. A suitable dosage amount of the composition of the present invention may vary depending on pharmaceutical formulation methods, administration methods, the patient's age, body weight, sex, pathogenic state, diet, administration time, administration route, an excretion rate and sensitivity for a used composition. The hyperthermia composition of the present invention includes a therapeutically effective amount of the present particle. The term “therapeutically effective amount” refers to an amount enough to treat a disease of interest and is generally administered with a daily dosage of 0.0001-100 mg/kg.

According to the conventional techniques known to those skilled in the art, the pharmaceutical composition of the present invention may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an elixir, a powder, a granule, a tablet and a capsule, and may further comprise a dispersion agent or a stabilizer.

The features and advantages of the present invention will be summarized as follows:

(i) The T1-T2 dual modal MRI contrast agent of the present invention primarily has a first layer-separating layer-second layer, and the first layer and second layer independently includes different type of contrast material.

(ii) The contrast agent of the present invention is a material which not only minimizes a reciprocal interference between T1 and T2 signal but also effectively generates both T1 and T2 signal in a single particle.

(iii) The T1-T2 dual-modal contrast agent of the present invention may generate both T1 and T2 signal and thus observe the signal complementarily, resulting in accurate diagnosis through reduction of misdiagnosis.

(iv) T1 and T2 MR imaging may be simultaneously obtained by simple operation within the same MR imaging device, enabling to remarkably reduce a diagnosis time and diagnosis cost.

(v) The particle constituting the T1-T2 dual-modal contrast agent of the present invention may be applied to hyperthermia and drug delivery systems.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Synthesis of Metal Oxide Nanoparticles (MFe₂O₄ (M=Mn, Fe, Co, Ni), ZnₓM₁₋ₓFe₂O (M=Mn, Fe; x=0.2, 0.3, 0.4, 0.8)) Used as T2 Contrast Material

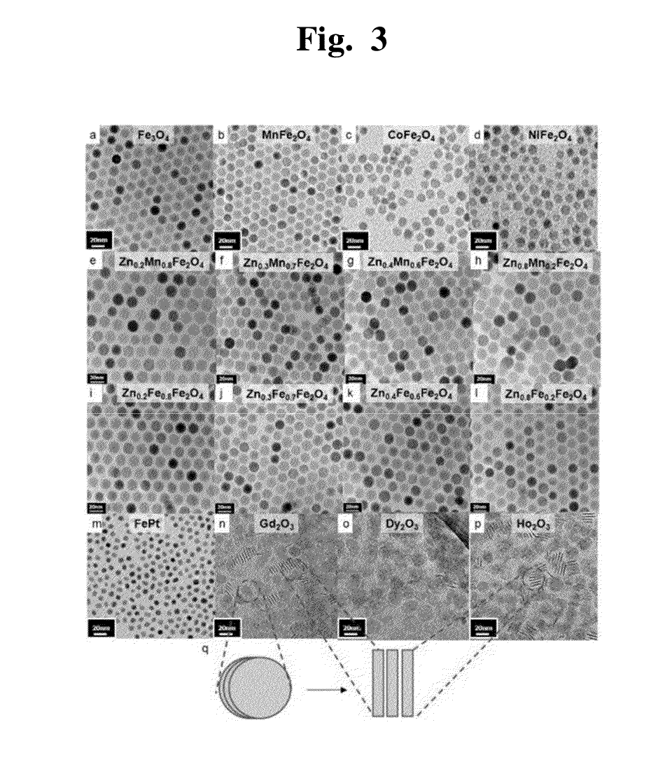

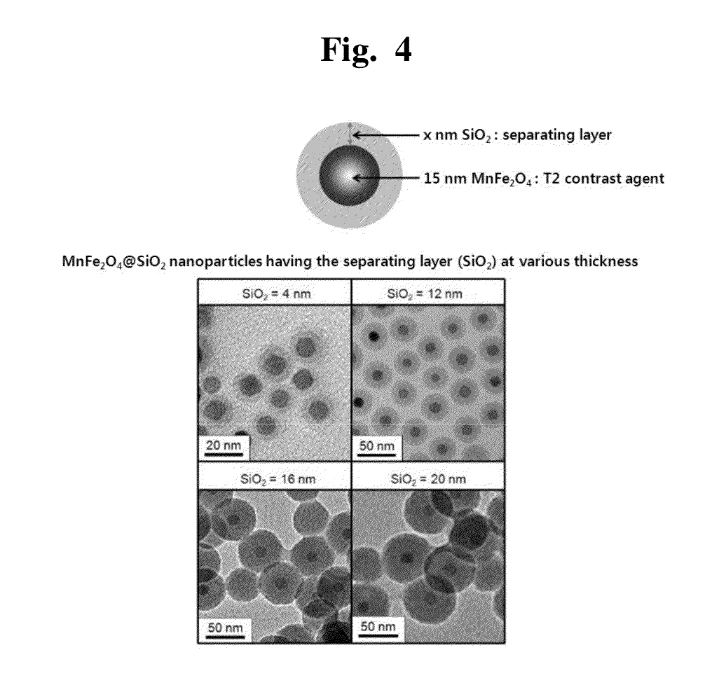

Metal oxide nanoparticles (MFe₂O₄ (M=Mn, Fe, Co, Ni), ZnₓMₓ₋₁Fe₂O₄ (M=Mn, Fe; x=0.2, 0.3, 0.4, 0.8) used as T2 contrast materials were produced according to the method described in Korean Pat. No. 0604975 filed by the present inventors. To prepare 15 nm-sized nanoparticles, Fe(acac)₃ (Aldrich, USA) and MCl₂ (M=Mn, Fe, Co, Ni, Zn; Aldrich, USA) as precursors of nanoparticles were mixed at an equivalence ratio of 2:1 and then added to 20 mL octylether solvent (Aldrich, USA) containing 0.1 M oleic acid (Aldrich, USA) and 0.1 M oleylamine (Aldrich, USA) as capping molecules. The mixture was incubated for 2 hrs at 290° C. under argon gas atmosphere. For preparation of Zn-containing metal oxides (ZnₓM₁₋ₓFe₂O₄), Fe(acac)₃ (Aldrich, USA) and ZnCl₂MCl₂ (M=Mn, Fe; Aldrich, USA) as precursors were mixed at an equivalence ratio of 2:1 whereas a ratio of ZnCl₂ to MCl₂ was controlled depending on composition of Zn (x=0.2, 0.3, 0.4, 0.8). It was demonstrated using TEM (Transmission Electron Microscopy) that all nanoparticles synthesized have a sphere shape with a size of 15 nm (FIGS. 3a-31). In FIG. 3, panel a represents Fe₃O₄; panel b represents MnFe₂O₄; panel c represents CoFe₂O₄; panel d represents NiFe₂O₄; panel e-h represent ZnₓMn₁₋ₓFe₂O₄ (x=0.2, 0.3, 0.4, 0.8); and panel i-l represent ZnₓFe₁₋ₓFe₂O₄ (x=0.2, 0.3, 0.4, 0.8).

### Example 2

### Synthesis of Metal Alloy Nanoparticles (FePt) Used as T2 Contrast Material

Metal alloy FePt nanoparticles used as T2 contrast materials were produced according to the method known to those skilled in the art (S. Sun et al. Journal of the American Chemical Society 2004, 126, 8394). As precursors of nanoparticles, 1 mmol of Fe(CO)₅ (Aldrich, USA) and 0.5 mmol of Pt(acac)₂ (Aldrich, USA) were added to dioctylether solvent (Aldrich, USA) containing 2 mmol oleic acid (Aldrich, USA) and 2 mmol oleylamine (Aldrich, USA) as capping molecules. The mixture was incubated for 1 hr at 200° C. under argon gas and further reacted for 2 hrs at 300° C. The nanoparticles synthesized were precipitated by excess ethanol and then isolated. The isolated nanoparticles were again dispersed in toluene, generating a colloid solution. All synthetic nanoparticles had a particle size of 6 nm with a sphere shape (FIG. 3m).

### Example 3

### Synthesis of Metal Oxide Nanoparticles (M₂O₃ (M=Gd, Ho, Dy)) Used as T1 Contrast Material

Metal oxide nanoparticles (M₂O₃ (M=Gd, Ho, Dy)) used as T1 contrast materials were produced according to the method described in Korean Pat. No. 0604975 filed by the present inventors. To prepare metal oxide nanoparticles with a sheet shape, MCl₂ (M=Gd, Ho, Dy; Aldrich, USA) as precursors of nanoparticles were added to octylether solvent (Aldrich, USA) containing 0.6 mmol oleic acid (Aldrich, USA) and 0.12 mmol oleylamine (Aldrich, USA) as capping molecules. The mixture was incubated for 2 hrs at 290° C. under argon gas atmosphere. By TEM (Transmission Electron Microscopy) analysis, it was demonstrated that all nanoparticles synthesized have a sheet shape with a size of about 1 nm and each Gd₂O₃, Dy₂O₃ and Ho₂O₃ has a diameter of 15, 25 and 20 nm (FIGS. 3n-3p). The practical shape of metal oxide nanoparticles synthesized is a sheet shape, but may be shown as a sphere or rod shape depending on observing angle (FIG. 3q). In other words, the sheet shape is observed as a sphere shape or a rod shape depending on lie or stand to electron beam, respectively.

### Example 4

### Synthesis of T2 Contrast Material (MnFe₂O₄@SiO₂) Coated with SiO₂ as the Separating Layer

The 15 nm-sized nanoparticles (1 mg) synthesized in Example 1, Igepal-50 (800 ml, Aldrich, USA) and cyclohexane (12 ml) were mixed with vigorous shaking (1,500 rpm). The solution was serially mixed with 30% ammonia water and TEOS (tetraethyl orthosilicate; Aldrich, USA) and then reacted without shaking for 3 days at room temperature. After stopping the reaction, the nanoparticles were mixed with 40 mL methanol and isolated using centrifugation (3,000 rpm, 5 min; a semi-diameter of centrifuge: 20 cm).

The thickness of SiO₂ used as the separating layer may be varied depending on the amount of silica alkoxide used. FIG. 4 represents TEM images of magnetic nanoparticles with a core size of 15 nm, MnFe₂O₄@SiO₂, coated with SiO₂ separating layer at various thickness (4, 12, 16, 20 nm).

### Example 5

### Synthesis of T1 Contrast Agent (MnFe₂O₄@SiO₂@Gd₂O(CO₃)₂.H₂O) with the Core-Separating Layer-Shell Structure in which T1 Contrast Material is Attached on the Separating Layer

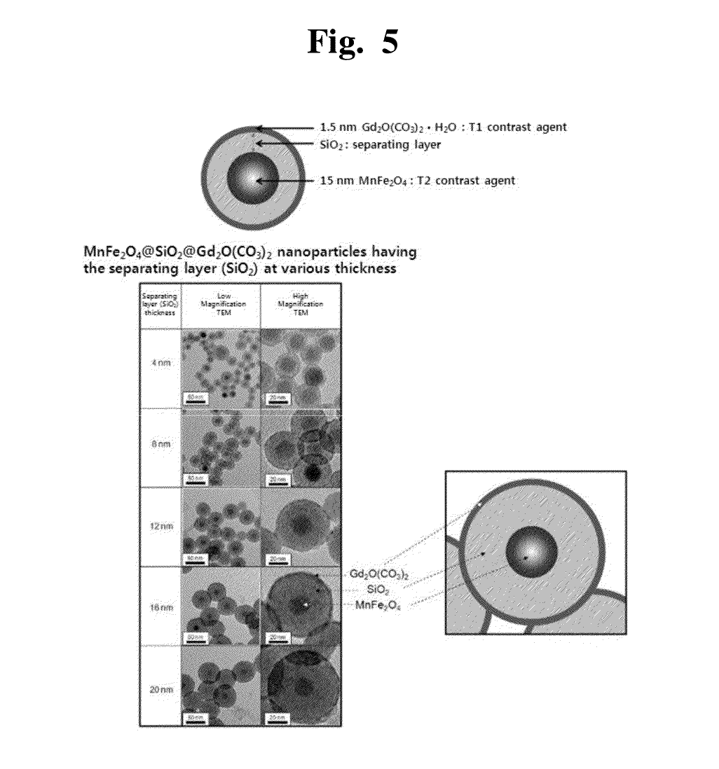

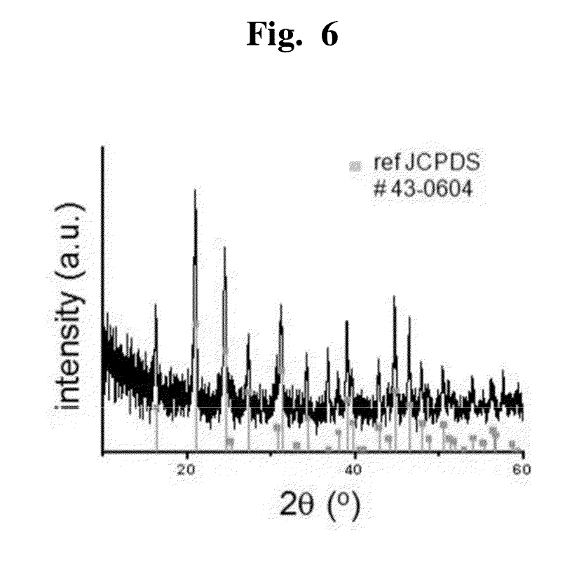

The 15 nm-sized nanoparticles (1 mg) synthesized in Example 4 were mixed with the solution containing 2 M ethanol urea and metal nitrate (MNO₃; M=Gd; 38 mM) and then reacted for 1 hr at 90° C. with shaking. After stopping the reaction, the nanoparticles were cooled to room temperature and mixed with acetone. Finally, the mixture was isolated using centrifugation (3,000 rpm, 10 min; a semi-diameter of centrifuge: 20 cm). In MnFe₂O₄@SiO₂@Gd₂O(CO₃)₂.H₂O nanoparticles synthesized, each T2 contrast material (MnFe₂O₄) and T1 contrast material (Gd₂O(CO₃)₂.H₂O) was located in the core and the shell, and SiO₂ layer was used as the separating layer. As shown in TEM images of T1-T2 contrast agent having the core-separating layer-shell structure in FIG. 5, all nanoparticles had 15 nm-sized core and 1.5 nm-sized shell. The thickness of SiO₂ used as the separating layer was varied in a range of from 4 nm to 20 nm (4, 8, 12, 16, 20 nm). FIG. 6 represents the structure of T1 contrast agent coating the surface of nanoparticle. In XRD (X-ray diffraction) graph, the crystalline structure of nanoparticle is consistent with Gd₂O(CO₃)₂.H₂O (JCPDS No. 43-0604).

### Example 6

### Comparison of T1 and T2 Signal of MnFe₂O₄@SiO₂@Gd₂O(CO₃)₂.H₂O Nanoparticles Depending on the Thickness of the Separating Layer (SiO₂)

MR imaging of the nanoparticles synthesized in Example 5 were taken using 1.5 T system (Acheiva 3.0; Philips Medical Systems. Best, the Netherlands) with a sense-flex-M coil. Magnetic resonance image is obtained using T1/2 FSE (fast spin echo sequence). The practical parameters are as follows: T1—slice thickness=1 mm, TE (echo time)=30 ms, TR (repetition time)=600 ms, FOV (field of view)=10×10 cm², number of excitation=2; T2—slice thickness=1 mm, TE=100 ms, TR=4,000 ms, FOV=10×10 cm², number of excitation=2.

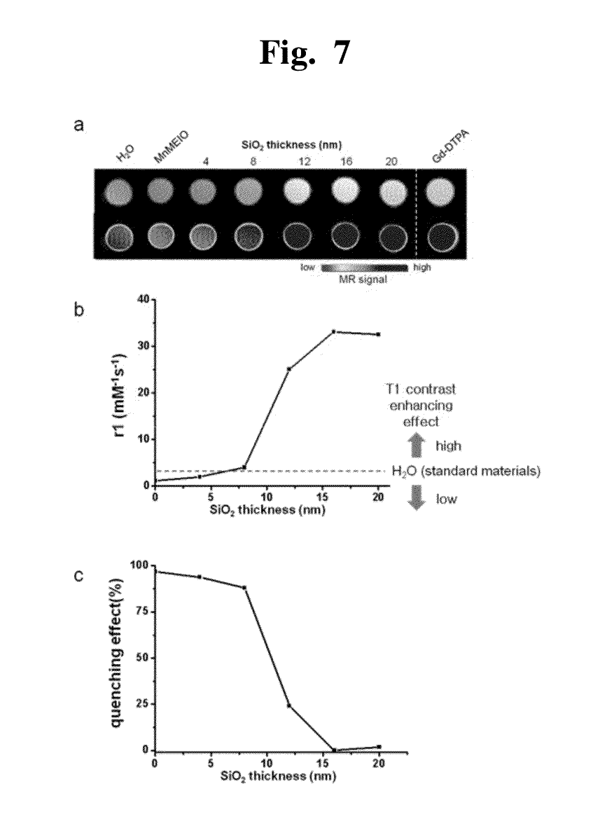

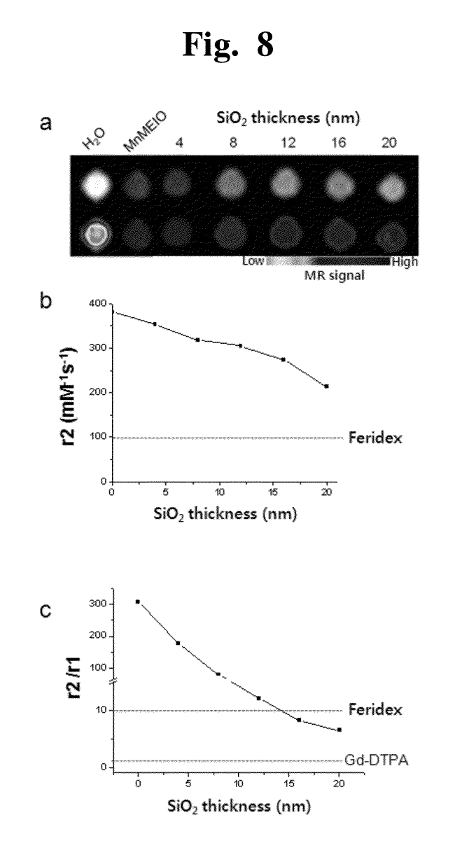

FIGS. 7-8 show the changes of T1 and T2 signal of MnFe₂O₄@SiO₂@Gd₂O(CO₃)₂.H₂O nanoparticles having 15 nm MnFe₂O₄ (T2 contrast material) as the core, 1.5 nm Gd₂O(CO₃)₂ (T1 contrast material) as the shell, and various SiO₂ thickness (0, 4, 8, 12, 16, 20 nm) as the separating layer. FIG. 7a is T1 MR image, and FIG. 7b is a graph representing T1 signal changes measured at FIG. 7a (nanoparticle concentration: 100 μg/ml (Gd)). In panel b, signal intensity of nanoparticles was represented as T1 relaxivity coefficient of nanomaterials compared with that of H₂O (standard material). r1 means T1 relaxation coefficient and T1 contrast effect is enhanced in proportion to the increase of r1 value. It was observed that T1 effect in T1 imaging is gradually enhanced depending on the increase of SiO₂ thickness, and is maximized at SiO₂ thickness of about 16 nm. T1 effect Gd₂O(CO₃)₂.H₂O was not observed in thin SiO₂ layer due to magnetic interference effect of MnFe₂O₄ (T2 contrast material) in the core. In addition, the signal of nanoparticles having thin SiO₂ layer was darker than that of H₂O. However, T1 signal was enhanced depending on the increase of SiO₂ thickness. T1 signal began to exhibit high contrast effect at SiO₂ thickness of about 8 nm and represented excellent contrast effect at SiO₂ thickness of 16 nm. This result demonstrates that the presence or absence of the separating layer and its thickness are important to simultaneously detect T1 and T2 contrast effect. FIG. 7c represents a relative T1 signal quenching effect depending on the thickness of the separating layer and is a graph comparing reduction of T1 signal with Gd-DTPA in the term of relaxation coefficient. On the other hand, FIGS. 8a-8b show T2 MR image and T2 signal comparative graph (nanoparticle concentration: 50 μg/ml (Mn+Fe), r2=T2 relaxivity coefficient). As shown in FIGS. 8a-8b, it was demonstrated that T2 contrast effect of nanoparticles represents a similar contrast effect when the thickness of the separating layer is in a range of 0-12 nm compared to core material (MnFe₂O₄) without the separating layer, whereas T2 contrast effect represents a gradual reduced contrast effect when the thickness of the separating layer is in a range of above 12 nm. However, all present nanoparticles entirely have T2 contrast effect much higher than H₂O without regard to the thickness of the separating layer.

### Example 7

### T1 and T2 Signal Analysis of T1-T2 Dual Modal MRI Contrast Agent with Core-Separating Layer-Shell Structure Having Various Compositions

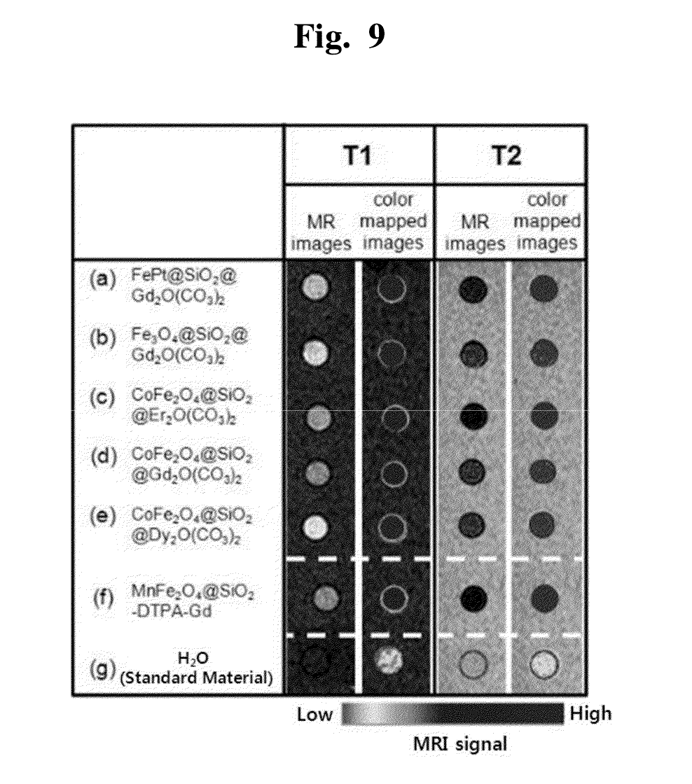

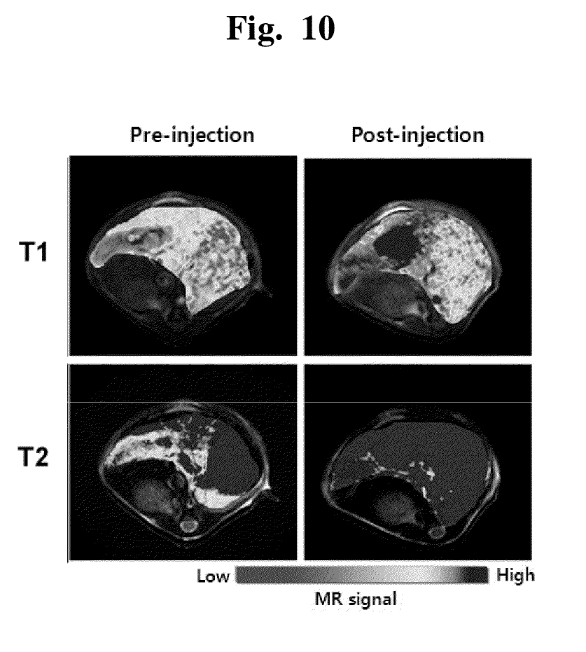

The contrast agent with core-separating layer-shell structure having various compositions was synthesized according to the method described in Example 5. SiO₂ with a thickness of 16 nm as the separating layer was introduced into all core-separating layer-shell-type contrast agents synthesized because it has excellent T1 or T2 contrast effect in the Example 6. In each core-separating layer-shell-type nanoparticle, various T2 contrast materials (metal oxides: 15 nm-sized Fe₃O₄, CoFe₂O₄, alloy: 6 nm-sized FePt) were used as the core and the separating layer (SiO₂) was coated with various T1 contrast materials (Gd₂O(CO₃)₂.H₂O, Dy₂O(CO₃)₂.H₂O, Er₂O(CO₃)₂.H₂O) with a thickness of 1.5 nm. For example, FePt@SiO₂@Gd₂O(CO₃)₂.H₂O, Fe₃O₄@SiO₂@Gd₂O(CO₃)₂.H₂O, CoFe₂O₄@SiO@Er₂O(CO₃)₂.H₂O, CoFe₂O₄@SiO₂@Gd₂O(CO₃)₂.H₂O, and CoFe₂O₄@SiO₂@Dy₂O(CO₃)₂.H₂O were prepared according to the methods described in the above Examples. T1 and T2 signal were analyzed under the almost same condition with MR imaging described in the Example 6. As shown in FIGS. 9a-9e, the contrast agent of the present invention significantly represented enhanced T1 and T2 signal where it has the separating layer without regard to its composition. The contrast agent of the present invention exhibited very bright signal in T1 image and very dark signal in T2 image as compared with H₂O (FIG. 9g), and had remarkable T1 and T2 contrast effect simultaneously in all contrast materials.

### Example 8

### T1 and T2 Signal Analysis of the Core-Separating Layer-Shell (Chelate)-Type MnFe₂O₄@SiO₂-DTPA-Gd Contrast Agent

MnFe₂O₄@SiO₂ was synthesized according to the method described in Example 5. One mg of nanoparticles (Mn+Fe) synthesized were mixed with 5 mL H₂O and further reacted with APTMS (3-aminopropyltrimethoxysilane: Aldrich, USA) for 1 hr at room temperature with shaking. After stopping the reaction, the nanoparticles were mixed with acetone isolated using centrifugation (3,000 rpm, 10 min; a semi-diameter of centrifuge: 20 cm). The isolated nanoparticles were reacted for 2 hrs with 50 mM EDC (1-ethyl-3-(3-dimethylaminopropyl)carbodiimide; Sigma, USA) and 5 mM sulfo-NHS (N-hydroxysulfosuccinimide; Pierce, USA) mixed with 0.5 mg of DTPA and H₂O. After reaction, the nanoparticles were isolated using a size exclusion column G-25. The isolated nanoparticles were mixed with 5 mg GdCl₂ and reacted for 2 hrs. The excessive salts were removed using a size exclusion column G-25. T1 and T2 signal were analyzed under the condition for MR imaging described in the Example 6. As shown in FIG. 9f, the contrast agent of the present invention represented enhanced T1 and T2 signal. It could be appreciated that the contrast agent of the present invention exhibited bright signal in T1 image and dark signal in T2 image as compared with H₂O.

### Example 9

### T1 and T2 Signal Analysis of the Core-Separating Layer-Shell (Polymer)-Type MnFe₂O₄@PS-b-PAA@Gd Contrast Agent

PS-b-PM (polystyrene-block-poly(acrylic acid) used in the Examples of the present invention was synthesized according to modification of the method described in Angew. Chem. Int. Ed. 2005, 44, 409. The procedure is as follows: t-Butyl Acrylate (Aldrich), PMDETA (methyldiethylenetriamine, Aldrich) and Cu(I)Br were mixed in a non-reactive state. Further, Methyl 2-bromopropionate was added to the mixture and then reacted for 1 hr at 60° C., generating PtBA [poly(tert-butyl acrylate)]. After obtaining PS (polystyrene) by the same method, two materials were mixed and then reacted for 3.5 hrs at 95° C. for polymerization. The synthesized PtBA-b-PS and p-toluenesulfonic acid were mixed in toluene and then refluxed for 20 hrs at 100° C., yielding PS-b-PM. The synthetic polymer was mixed with nanoparticles synthesized in the Example 1 and DMF, and gradually added with distilled water. The particles were isolated using dialysis. The nanoparticles isolated were adjusted to pH 7 and isolated using centrifugation. Finally, the nanoparticles were reacted with GdCl₃ and isolated using centrifugation. T1-T2 nanoparticles altering GdCl₃ to DyCl₃, ErCl₃, HoCl₃ or TbCl₃ could be fabricated using the same method. T1 and T2 signal were analyzed under the condition for MR imaging described in the Example 6. As shown in FIG. 9g, the contrast agent of the present invention represented enhanced T1 and T2 signal. It could be appreciated that the contrast agent of the present invention exhibited very bright signal in T1 image and very dark signal in T2 image as compared with H₂O.

### Example 10

### T1 and T2 Signal Analysis of the Core-Separating Layer-Shell (Protein)-Type MnFe₂O₄@serum albumin@Gd Contrast Agent

MnFe₂O₄@serum albumin used in the Examples of the present invention was synthesized according to the method described in Korean Pat. No. 10-0713745. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of serum albumin (Aldrich, USA) was dissolved in 1 mL of deionized water and mixed with the precipitates, synthesizing nanoparticles coated with SA. Finally, non-reactive SA was removed using a Sephacryl S-300 column (GE healthcare, USA), obtaining pure SA-coated water-soluble nanoparticles. The isolated nanoparticles were reacted for 2 hrs with 50 mM EDC (Sigma, USA) and 5 mM sulfo-NHS (Pierce, USA) mixed with 0.5 mg of DTPA and H₂O. After reaction, the nanoparticles were isolated using a size exclusion column G-25. The isolated nanoparticles were mixed with 5 mg GdCl₂ and reacted for 2 hrs. The excessive salts were removed using a size exclusion column G-25. T1-T2 nanoparticles altering GdCl₃ to DyCl₃, ErCl₃, HoCl₃ or TbCl₃ could be fabricated using the same method. T1 and T2 signal were analyzed under the condition for MR imaging described in the Example 6. As shown in FIG. 9h, the contrast agent of the present invention represented enhanced T1 and T2 signal. It could be appreciated that the contrast agent of the present invention exhibited bright signal in T1 image and dark signal in T2 image as compared with H₂O.

### Example 11

### Analysis of T1 and T2 Signal in Liver Tissue Using the Core-Separating Layer-Shell-Type MnFe₂O₄@SiO₂@Gd₂O(CO₃)₂.H₂Contrast Agent

The contrast ability for liver was investigated using MRI with MnFe₂O₄@SiO₂@Gd₂O(CO₃)₂.H₂O contrast agent synthesized in the Example 5. MnMEIO@SiO₂@Gd₂O(CO₃)₂.H₂O was intravenously injected into the tail of rats at a concentration of 1 mg (Mn+Fe)/kg. Magnetic resonance imaging (MRI) was measured at 1 hr pre- and post-injection of nanoparticles. To measure MRI, T1 and T2 signal were measured using 3.0 T MRI (achieva XT, Philips, Netherland) and the practical parameters are as follows; T1—TR (repetition time)=4,000 ms, TE (echo time)=10 ms, FOV (field of view)=60 mm, matrix=256×256, slice thickness=2 m, number of excitation=1; T2—TR=4,000 ms, TE=80 ms, FOV=6 mm.

As shown in FIG. 10, both T1 and T2 signal in liver tissue were detected at post-injection higher than at pre-injection. Therefore, it could be appreciated that the present nanoparticles can effectively contrast liver tissue in both imaging modes of MRI.

### Example 12

### Analysis of T1 and T2 Signal in Cancer Tissue Using the Core-Separating Layer-Shell-Type MnFe₂O₄@SiO₂@Gd₂O(CO₃)₂Contrast Agent

The contrast ability for cancer was investigated using MRI with MnFe₂O₄@SiO₂@Gd₂O(CO₃)₂ contrast agent synthesized in the Example 5. MnMEIO@SiO₂@Gd₂O(CO₃)₂.H₂O was intravenously injected into the tail of rats at a concentration of 5 mg (Mn+Fe)/kg. Magnetic resonance imaging (MRI) was measured at 1 hr pre- and post-injection of nanoparticles. MRI was measured under the condition suggested in the Example 11.

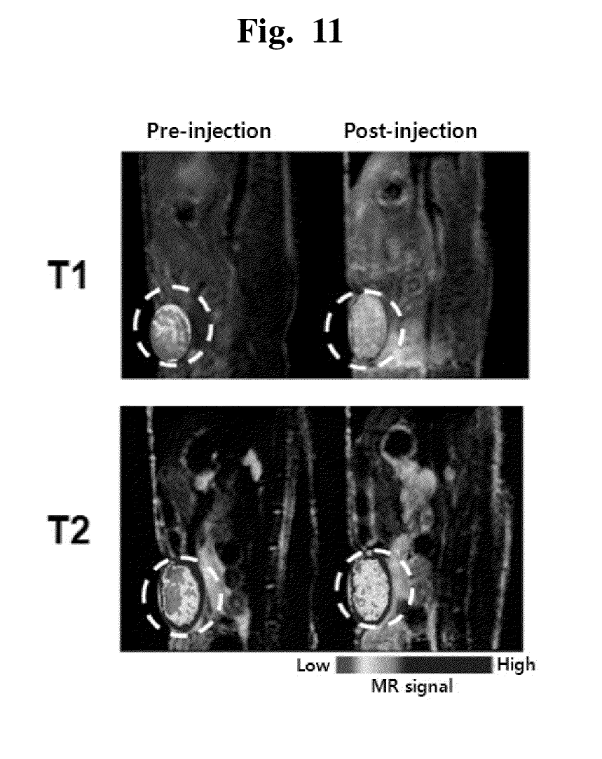

As shown in FIG. 11, both T1 and T2 signal in cancer tissue were detected at post-injection higher than at pre-injection. Imaging for cancer tissue using nanoparticle is widely classified into two types: (a) an active targeting and (b) a passive targeting. The active targeting recognizes cancer cells using attachment of a targeting biomolecule such as a tumor-specific antibody or peptide, whereas the passive targeting selectively recognizes cancer cells based on the fact that cancer has loose tissue compared with normal tissue. In general, nanoparticles with a size of several-200 nm are not penetrated into cell. However, these nanoparticles may be used in selective cancer diagnosis because they can be accumulated by biological injection within cancer cell of which the blood vessel is loose tissue. Therefore, it could be appreciated that nanoparticles represented in two suggestive images effectively contrast cancer tissues in MRI.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A T1-T2 dual-modal MRI (magnetic resonance imaging) contrast agent having core-shell structure, comprising (a) a core containing a T2 contrast material; (b) a shell containing a T2 contrast material; and (c) a separating layer, located between the core and the shell, wherein the core and shell comprise a different type of the contrast materials from each other, wherein the ratio of an average thickness of the separating layer and an average size of the core ranges from 8:15 to 4:3, and wherein the separating layer comprises:
(i) a metal chalcogen, MₓA_{y} [M=one or more elements selected from the group consisting of Group 2 metal elements (Be, Mg, Ca, Sr, Ba), Group 13 metal elements (Al, In, Tl), Group 14 metal elements (Si, Ge, Sn, Pb), Group 15 metal elements (As, Sb, Bi), transition metal elements (Sc, Ti, V, Zn, Y, Zr, Nb, Mo), Lanthanide metal elements (Ce, Pr, Nd, Pm, Sm, Eu, Lu) and Actinide metal elements; A is one or more elements selected from the group consisting of O, S, Se and Te; 0<x≦16, 0<y≦8], or a multi-component hybrid structure thereof; (ii) a ceramic material; (iii) a polymer; (iv) a protein; (v) a carbohydrate; or (vi) a multi-component hybrid structure thereof, and wherein the separating layer is not a liposomal membrane.

2. A method for providing T1 and T2 images of an internal region of a patient simultaneously without a reciprocal interference between T1 signal and T2 signal, comprising:
(a) administering to the patient a diagnostically effective amount of the T1-T2 dual-modal MRI (magnetic resonance imaging) contrast agent according to claim 1; and
(b) scanning the patient using a magnetic resonance imaging to obtain visible T1 and T 2 images of the region.

3. A method for enabling a reciprocal interference between T1 signal and T2 signal from a T1-T2 dual-modal MRI (magnetic resonance imaging) contrast agent to be prevented, comprising:
(a) designing the T1-T2 dual-modal MRI contrast agent to have a core-shell structure comprising (a-1) a core containing a T2 contrast material; (a-2) a shell containing a T2 contrast material; and (a-3) a separating layer, located between the core and the shell, wherein the core and shell comprise a different type of the contrast materials from each other; wherein the separating layer comprises: (i) a metal chalcogen, MₓA_{y} [M=one or more elements selected from the group consisting of Group 2 metal elements (Be, Mg, Ca, Sr, Ba), Group 13 metal elements (Al, In, Tl), Group 14 metal elements (Si, Ge, Sn, Pb), Group 15 metal elements (As, Sb, Bi), transition metal elements (Sc, Ti, V, Zn, Y, Zr, Nb, Mo), Lanthanide metal elements (Ce, Pr, Nd, Pm, Sm, Eu, Lu) and Actinide metal elements; A is one or more elements selected from the group consisting of O, S, Se and Te; 0<x≦16, 0<y≦8], or a multi-component hybrid structure thereof; (ii) a ceramic material; (iii) a polymer; (iv) a protein; (v) a carbohydrate; or (vi) a multi-component hybrid structure thereof, and wherein the separating layer is not a liposomal membrane; and
(b) determining an average thickness of the separating layer and an average size of the core to allow that the ratio of the average thickness of the separating layer and the average size of the core ranges from 8:15 to 4:3, such that the reciprocal interference between T1 signal and T2 signal from the T1-T2 dual-modal MRI contrast agent is prevented by the separating layer in an internal region of a patient.

4. The method according to claim 3, wherein the method further comprises the steps of (c) preparing the T1-T2 dual-modal MRI contrast agent designed; (d) administering to the patient a diagnostically effective amount of the T1-T2 dual-modal MRI contrast agent; and (e) scanning the patient using a magnetic resonance imaging to obtain visible T1 and T 2 images of the region, such that the visible T1 and T 2 images is obtained with no reciprocal interference between T1 signal and T2 signal from the T1-T2 dual modal MRI contrast agent.

5. A method for preventing a reciprocal interference between T1 signal and T2 signal from a T1-T2 dual-modal MRI (magnetic resonance imaging) contrast agent, comprising:
(a) designing the T1-T2 dual-modal MRI contrast agent to have a core-shell structure comprising (a-1) a core containing a T2 contrast material; (a-2) a shell containing a T2 contrast material; and (a-3) a separating layer, located between the core and the shell, wherein the core and shell comprise a different type of the contrast materials from each other; wherein the separating layer comprises: (i) a metal chalcogen, MₓA_{y} [M=one or more elements selected from the group consisting of Group 2 metal elements (Be, Mg, Ca, Sr, Ba), Group 13 metal elements (Al, In, Tl), Group 14 metal elements (Si, Ge, Sn, Pb), Group 15 metal elements (As, Sb, Bi), transition metal elements (Sc, Ti, V, Zn, Y, Zr, Nb, Mo), Lanthanide metal elements (Ce, Pr, Nd, Pm, Sm, Eu, Lu) and Actinide metal elements; A is one or more elements selected from the group consisting of O, S, Se and Te; 0<x≦16, 0<y≦8], or a multi-component hybrid structure thereof; (ii) a ceramic material; (iii) a polymer; (iv) a protein; (v) a carbohydrate; or (vi) a multi-component hybrid structure thereof, and wherein the separating layer is not a liposomal membrane; and
(b) determining an average thickness of the separating layer and an average size of the core to allow that the ratio of the average thickness of the separating layer and the average size of the core ranges from 8:15 to 4:3, such that the reciprocal interference between T1 signal and T2 signal from the T1-T2 dual-modal MRI contrast agent is prevented by the separating layer in an internal region of a patient.

6. The method according to claim 5, wherein the method further comprises the steps of (b) preparing the T1-T2 dual-modal MRI contrast agent designed; (c) administering to the patient a diagnostically effective amount of the T1-T2 dual-modal MRI contrast agent; and (d) scanning the patient using a magnetic resonance imaging to obtain visible T1 and T 2 images of the region, such that the visible T1 and T 2 images is obtained with no reciprocal interference between T1 signal and T2 signal from the T1-T2 dual-modal MRI contrast agent.
